(19) United States
(12) Patent Application Publication
(10) Pub. No.: US 2024/0238552 A1
(43) Pub. Date: Jul. 18, 2024

(54) METHODS AND SYSTEMS FOR MANUAL VENTILATION

(71) Applicant: GE Precision Healthcare LLC, Waukesha, WI (US)

(72) Inventors: Elvin Cyriac, Bengaluru (IN); Nandakumar Jegathala Raju, Bengaluru (IN); Hariprasad A. Kodancha, Bengaluru (IN); Tom J. Haggblom, Uusimaa (FI)

(21) Appl. No.: 18/622,295

(22) Filed: Mar. 29, 2024

Related U.S. Application Data
(63) Continuation-in-part of application No. 18/147,235, filed on Dec. 28, 2022.

(51) Int. Cl. A61M 16/20; A61M 16/00

(52) U.S. Cl. CPC A61M 16/202; A61M 16/0078; A61M 2016/0027; A61M 2205/3337; A61M 2205/3344

(57) ABSTRACT

A method for controlling a medical ventilator including a manual ventilation circuit, a mechanical ventilation circuit, and a patient circuit, includes: obtaining an encoder value based on a pressure limit of a mechanical adjustable pressure limit valve (APLV) in the manual ventilation circuit; controlling a pressure limit of an electro-pneumatic APLV in the manual ventilation circuit based on the encoder value, the electro-pneumatic APLV being pneumatically connected in parallel to the mechanical APLV.

### CROSS-REFERENCE TO RELATED APPLICATION

This application is a continuation-in-part of U.S. patent Application No. 18/147,235 filed on Dec. 28, 2022 in the U.S. patent and Trademark Office.

### FIELD

Embodiments of the subject matter disclosed herein relate to assisted ventilation of a subject.

### BACKGROUND

During an event where ventilation support is demanded for a subject, such as a patient, undergoing a surgery or a procedure which requires anesthetization, a ventilation system may be used to provide requisite pulmonary gas exchanges to sustain life. The ventilation system may have a relatively complex configuration for delivering oxygen to and removing carbon dioxide from the subject's lungs and may rely on microprocessor-based control of sensors, valves, flow rate controllers, and various other components. The subject may thereby be mechanically ventilated and flow of gases to and from the subject may be monitored and controlled by the ventilation system.

For example, a Positive End Expiratory Pressure (PEEP) may be delivered during mechanical ventilation where a positive pressure is maintained in the subject's airways at the end of an exhalation that is greater than atmospheric pressure. By applying PEEP, the alveoli in the lungs of the patient may be maintained open, which may otherwise collapse at the end of a respiratory cycle.

The ventilation system may also include a manual ventilation circuit. For example, the manual ventilation circuit may include a bag and mask for operation of the ventilation system in a bag mode (e.g., a manual ventilation mode). Manual ventilation also plays a vital role during patient intubation and weaning processes. In conventional bag mode operation, the bag may be configured with an adjustable pressure limiting (APL) valve. The APL valve controls a maximum pressure of the bag during inspiration (e.g., inhalation) of the subject and enables venting of excess pressure. When providing ventilation to the subject via the bag having the APL valve, determination of a volume of gases delivered to the patient upon bag compression may be challenging and provision of PEEP is precluded. The effectiveness of the manual ventilation and maintaining PEEP in bag mode is solely dependent on the experience and skill of the anesthesiologist.

### BRIEF DESCRIPTION

According to an aspect of the disclosure, a method for controlling a medical ventilator including a manual ventilation circuit, a mechanical ventilation circuit, and a patient circuit, may include: obtaining an encoder value based on a pressure limit of a mechanical adjustable pressure limit valve (APLV) in the manual ventilation circuit; controlling a pressure limit of an electro-pneumatic APLV in the manual ventilation circuit based on the encoder value, the electro-pneumatic APLV being pneumatically connected in parallel to the mechanical APLV.

According to another aspect of the disclosure, a medical ventilator may include: a manual ventilation circuit configured to provide manual ventilation to a patient, the manual ventilation circuit may include: a bag configured to be operated by a user; a mechanical adjustable pressure limit valve (APLV); a electro-pneumatic APLV pneumatically connected in parallel with the mechanical APLV; an encoder configured to output an encoder value based on a pressure limit of the mechanical APLV; and a shutoff valve between the bag and the mechanical APLV; a mechanical ventilation circuit configured to provide mechanical ventilation to a patient through bellows; a patient circuit configured to deliver gas to and remove gas from a patient's lungs; a memory storing instructions; and at least one processor configured to execute the instructions to: control a pressure limit of the electro-pneumatic APLV based on the encoder value.

According to yet another aspect of the disclosure, a medical ventilator system may include: a manual ventilation circuit including: a bag configured to be operated by a user; and a user adjustable encoder configured to output an encoder value based on a pressure limit of a first adjustable pressure limit valve (APLV) in the manual ventilation circuit; a mechanical ventilation circuit may include an electronically controlled mechanical ventilator; a patient circuit configured to deliver gas to and remove gas from a patient's lungs; a first flow path provided between the manual ventilation circuit and the patient circuit; a second flow path provided between the mechanical ventilation circuit and the patient circuit; a first electronically controlled two-way valve having a first position providing the first flow path and a second position providing the second flow path; a memory storing instructions; and at least one processor configured to execute the instruction to: control a maximum pressure limit in the first flow path based on the encoder value; control the first two-way valve based on the encoder value.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an example of a medical system, including a ventilation system for providing respiratory support to a subject.

FIG. 2 shows a block diagram of the medical system of claim 1.

FIG. 3 shows a block diagram of the ventilation system of the medical system of FIGS. 1-2.

FIG. 4 shows a schematic diagram of a manual ventilation circuit adapted with a manual ventilation PEEP delivery system.

FIG. 5 shows a pneumatic diagram of a ventilation system configured with a manual ventilation PEEP delivery system, according to an embodiment.

FIGS. 6A-6C show operating configurations of a pressure regulating valve with solenoid actuation included in the manual ventilation PEEP delivery system of FIG. 6.

FIG. 7 shows an example of a method for operating a ventilation system configured with a manual ventilation PEEP delivery system.

FIG. 8 shows an example of a method for delivering PEEP during manual ventilation.

FIG. 9 shows examples of variations in operating parameters of a ventilation system configured with a manual ventilation PEEP delivery system.

FIG. 10 shows a diagram of a ventilation system configured with a manual ventilation PEEP delivery system, according to an embodiment.

FIG. 11 shows a pneumatic diagram of a ventilation system configured with a manual ventilation PEEP delivery system, according to an embodiment.

FIG. 12 shows a control diagram of a ventilation system 1000 configured with a manual ventilation PEEP delivery system, according to an embodiment.

FIG. 13, shows a relationship between a mechanical APL valve 1010 and encoder 1011, according to an embodiment.

FIG. 14 is a flowchart of a process of controlling a ventilation system configured with a manual ventilation PEEP delivery system, according to an embodiment.

FIG. 15 shows a diagram of a belowless ventilation system configured with a manual ventilation PEEP delivery system, according to an embodiment.

### DETAILED DESCRIPTION

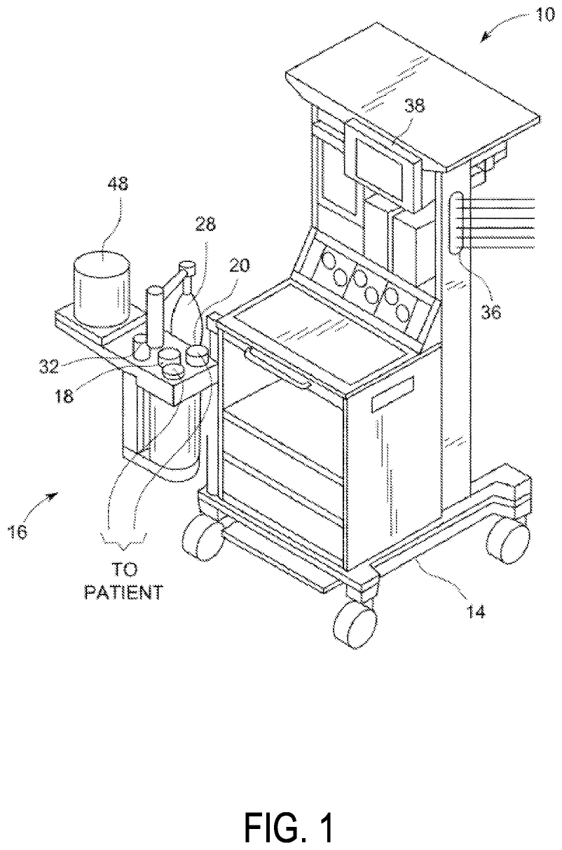

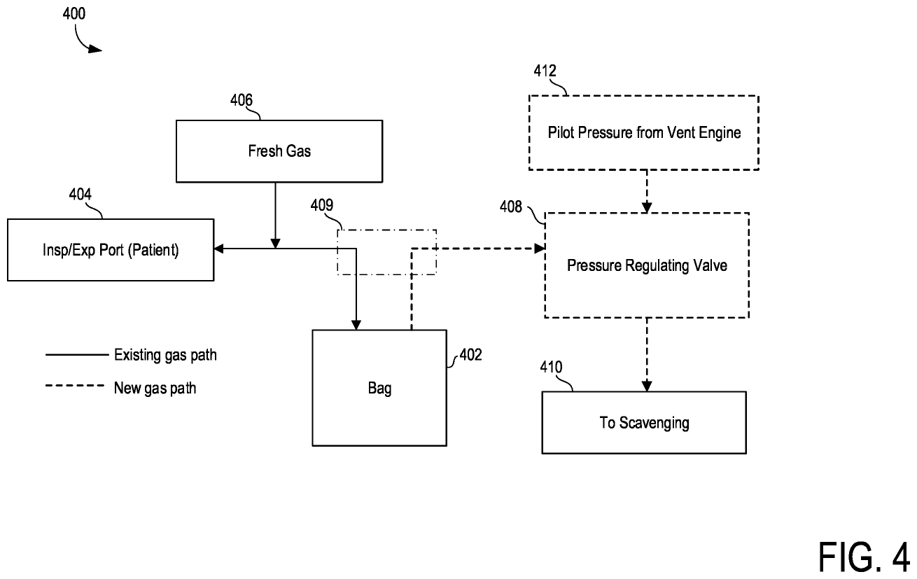

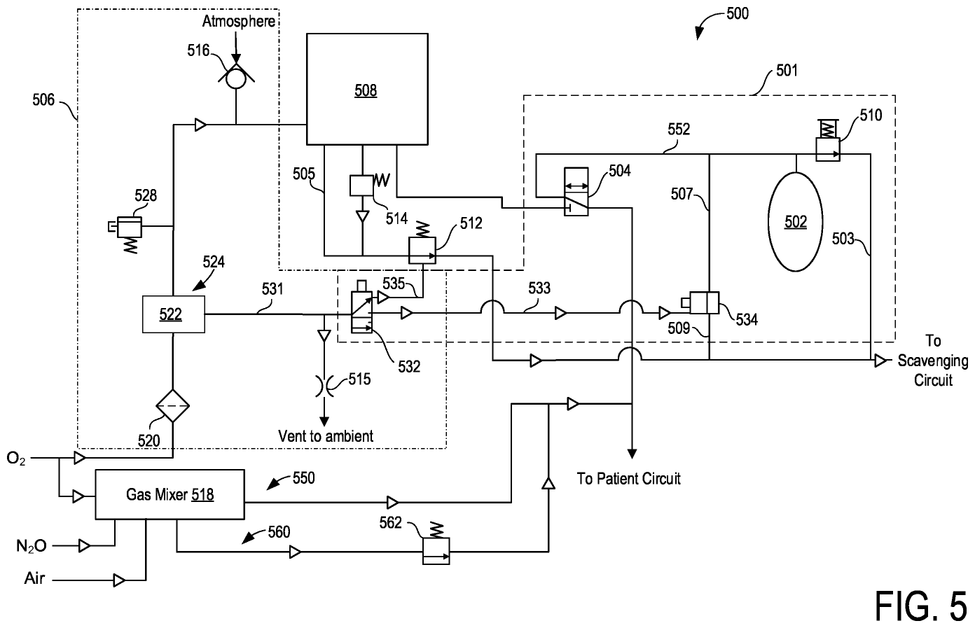

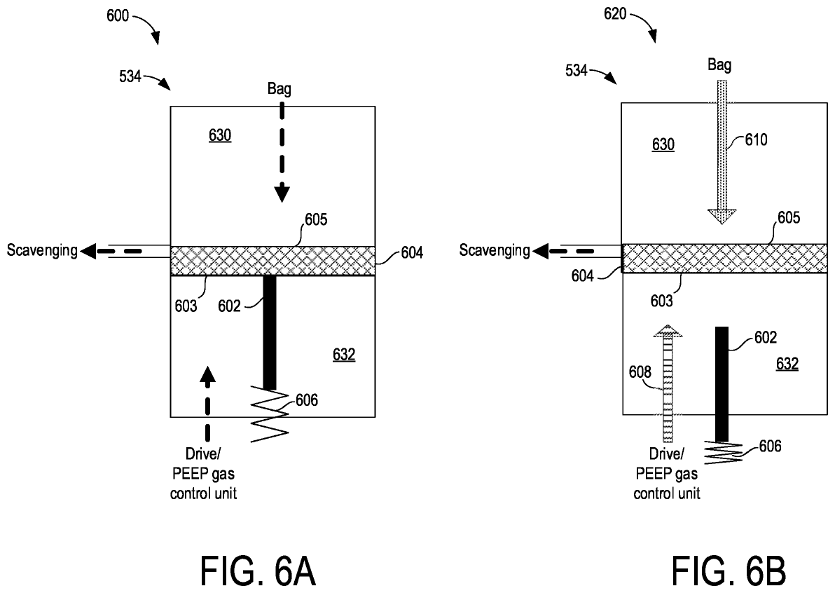

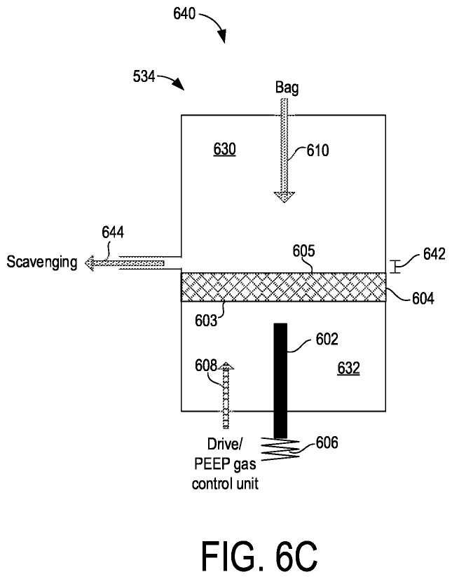

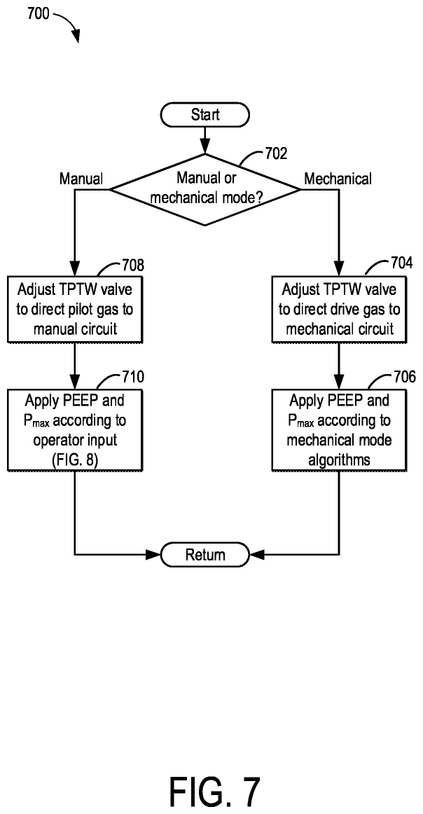

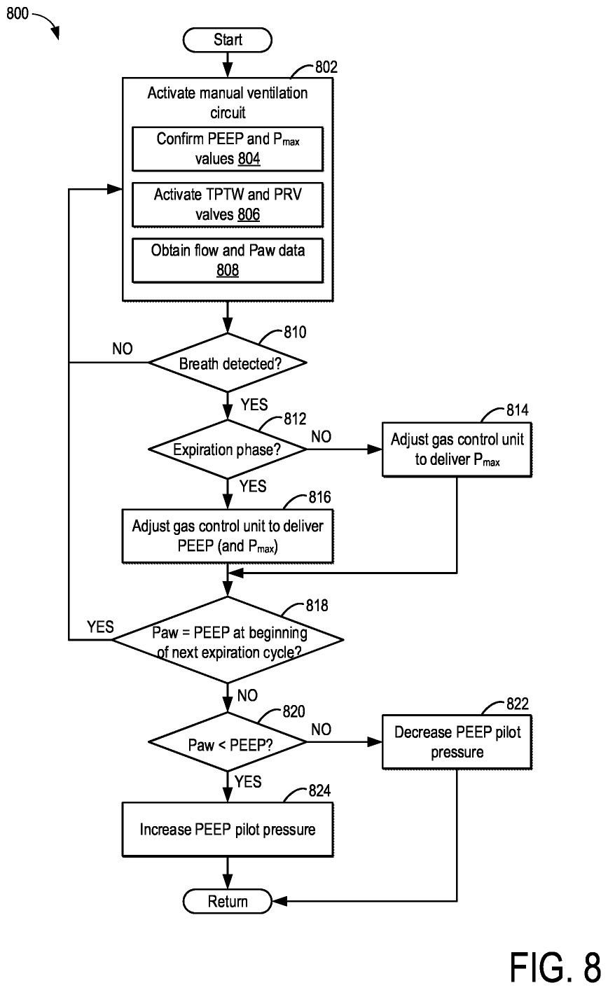

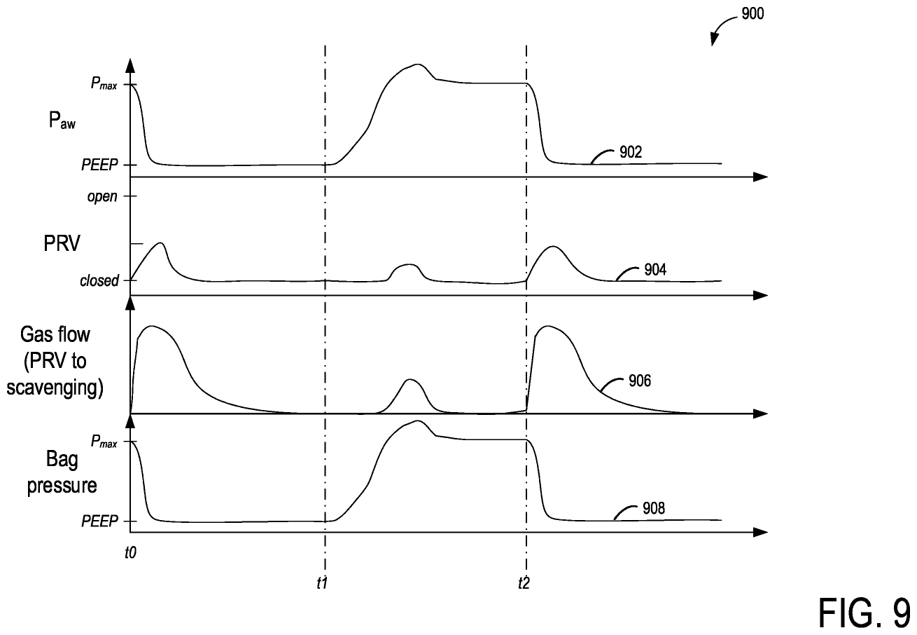

The following description relates to various embodiments of a ventilation system. In one example, the ventilation system may be configured to be operated in more than one mode, including a mechanical mode, where ventilation assistance is provided by a controller of the ventilation system, and a manual mode, where ventilation assistance is provided by a bag that is manually actuated. An example of a medical system with a ventilation system enabling both mechanical and manual ventilation support is shown in FIGS. 1-3. In order to provide a positive end expiration pressure (PEEP) delivery system for enabling control of PEEP during operation of the ventilation system in the manual mode, at least a manual ventilation portion of the ventilation system may be configured with additional components, as shown in FIG. 4. A pneumatic diagram of the ventilation showing incorporation of the PEEP delivery system for enabling control of PEEP is illustrated in FIG. 5, where the PEEP delivery system may rely on a solenoid-actuated and pneumatically-controlled pressure regulating valve for channeling gas flow from bag to scavenging according to the operating status of the ventilation system during manual ventilation. An example of the solenoid-actuated pressure regulating valve is shown in different operating configurations in FIGS. 6A-6C. An example of a method for operating a ventilation system equipped with a PEEP delivery system is shown in FIG. 7 and an example of a method for providing PEEP via the PEEP delivery system during manual ventilation is depicted in FIG. 8. Illustrative examples of how operating parameters of a ventilation system including the PEEP delivery system for manual ventilation are shown in FIG. 9 in a prophetic graph.

Before further discussion of the approach for enabling PEEP during manual ventilation, a general description of a medical system configured to provide ventilation support is provided. The figures illustrate diagrams of the functional blocks of various embodiments. The functional blocks are not necessarily indicative of the division between hardware circuitry. Thus, for example, one or more of the functional blocks (e.g., processors or memories) may be implemented in a single piece of hardware (e.g., a general purpose signal processor or a block or random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings.

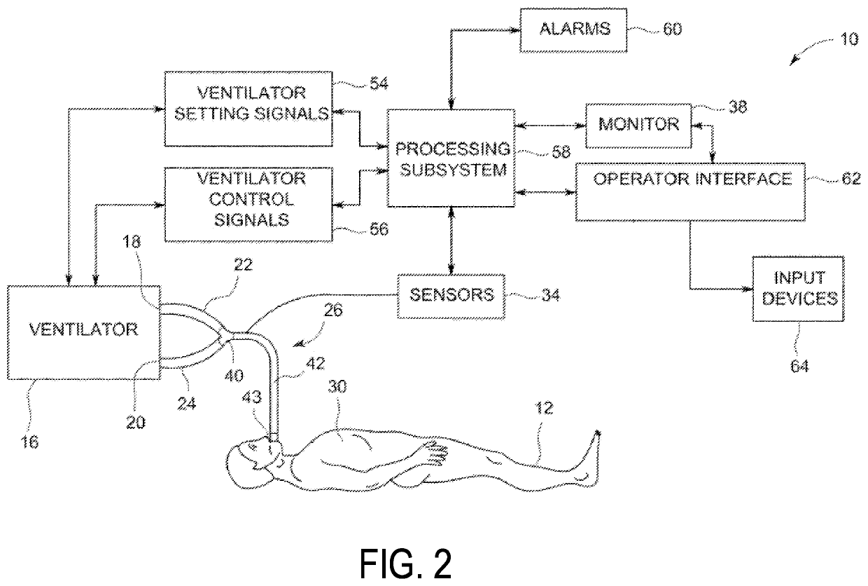

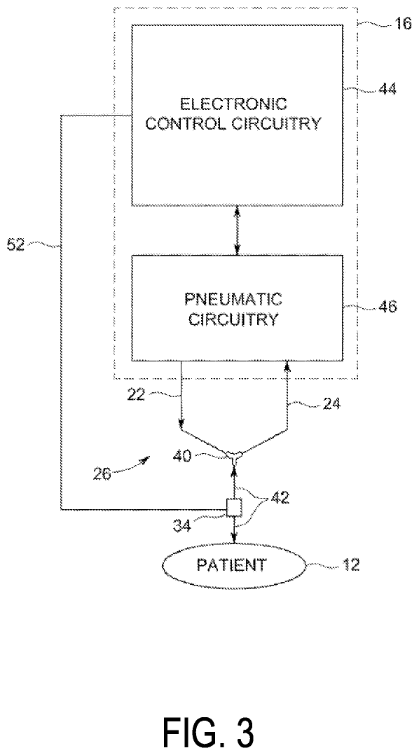

For example, one embodiment of a medical system 10 is shown in FIGS. 1-3, which may include an anesthesia machine 14 with a ventilator 16. In one example, the medical system 10 may be a ventilation system. The ventilator may have suitable connectors 18, 20 for connecting to an inspiratory branch 22 and expiratory branch 24 of a breathing circuit 26 leading to a patient 12, where the inspiratory branch 22, the expiratory branch 24, the breathing circuit 26, and the patient 12 are depicted in FIGS. 2 and 3. The ventilator 16 and breathing circuit 26 may cooperate to provide breathing gases to the patient 12 via the inspiratory branch 22 and to receive gases expired by the patient 12 via the expiratory branch 24.

The ventilator 16 may also be provided with a manual resuscitator 28, as shown in FIG. 1, for manually ventilating the patient 12. In one example, the manual resuscitator 28 may be a bag valve mask (BVM), which may also be referred to as a bag. For example, the manual resuscitator 28 may be filled with breathing gases, such as oxygen, anesthesia gases, etc., and be manually squeezed by a clinician (not shown) to provide the breathing gases to the patient 12. Using this manual resuscitator 28, or “bagging the patient,” enables clinicians to manually and/or immediately control delivery of the breathing gases to the patient 12. The clinician may also sense conditions in the respiration and/or lungs 30 (as shown in FIG. 2) of the patient 12 according to a sensory feedback during manipulation of the manual resuscitator 28, and adjust manual ventilation accordingly. The ventilator 16 may also provide a bag-to-ventilation (BTV) switch 32 for switching and/or alternating between manual and automated (e.g., mechanical) ventilation when the manual resuscitator 28 is provided.

The ventilator 16 may receive inputs from sensors 34, as shown in FIG. 2, associated with the ventilator 16 at a processing terminal 36 of FIG. 2 for subsequent processing thereof. The processed inputs may be displayed on a monitor 38. Representative data received from the sensors 34 may include, for example, inspiratory time (T_{I}), expiratory time (T_{E}), natural exhalation time (T_{EXH}), respiratory rates (f), I:E ratios, positive end expiratory pressure (PEEP), fractional inspired oxygen (F_{I}O₂), fractional expired oxygen (F_{E}O₂), breathing gas flow (F), tidal volumes (V_{T}), temperatures (T), airway pressures (P_{aw}), arterial blood oxygen saturation levels (SₐO₂), blood pressure information (BP), pulse rates (PR), pulse oximetry levels (SₚO₂), exhaled CO₂ levels (F_{ET}CO₂), concentration of inspired inhalation anesthetic agent (C_{I} agent), concentration of expired inhalation anesthetic agent (C_{E} agent), arterial blood oxygen partial pressure (PₐO₂), arterial carbon dioxide partial pressure (PₐCO₂), and the like.

With particular reference now to FIGS. 2-3, the ventilator 16 provides breathing gases to the patient 12 via the breathing circuit 26. Accordingly, the breathing circuit 26 includes the inspiratory branch 22 and expiratory branch 24. In one embodiment, one end of each of the inspiratory branch 22 and expiratory branch 24 is connected to the ventilator 16, while the other ends thereof are connected to a Y-connector 40, which can then connect to the patient 12 through a patient branch 42. An interface 43 also may be provided to secure the patient's 12 airways to the breathing circuit 26 and/or prevent gas leakage out thereof.

As shown in FIG. 3, the ventilator 16 may also include electronic control circuitry 44 and/or pneumatic circuitry 46. In particular, various pneumatic elements of the pneumatic circuitry 46 may provide breathing gases to the lungs 30 of the patient 12 through the inspiratory branch 22 of the breathing circuit 26 during inhalation. Upon exhalation, the breathing gases may be discharged from the lungs 30 of the patient 12 and into the expiratory branch 24 of the breathing circuit 26. This process can be iteratively enabled by the electronic control circuitry 44 and/or pneumatic circuitry 46 in the ventilator 16, which can establish various control parameters, such as the number of breaths per minute to administer to the patient 12, tidal volumes (V_{T}), maximum pressures, etc., that can characterize the mechanical ventilation that the ventilator 16 supplies to the patient 12. As such, the ventilator 16 may be microprocessor-based and operable in conjunction with a suitable memory to control pulmonary gas exchanges in the breathing circuit 26 connected to, and between, the patient 12 and the ventilator 16.

The various pneumatic elements of the pneumatic circuitry 46 may also include a source of pressurized gas (not shown), which can operate through a gas concentration subsystem (not shown) to provide the breathing gases to the lungs 30 of the patient 12. The pneumatic circuitry 46 may provide the breathing gases directly to the lungs 30 of the patient 12, as may be used in a chronic and/or critical care application, or the pneumatic circuitry 46 may provide a driving gas to compress a bellows 48 (as shown in FIG. 1) containing the breathing gases. In turn, the bellows 48 may supply the breathing gases to the lungs 30 of the patient 12, such as in an anesthesia application. In either case, the breathing gases may iteratively pass from the inspiratory branch 22, to the Y-connector 40 and to the patient 12, and then back to the ventilator 16 via the Y-connector 40 and expiratory branch 24.

In the embodiment illustrated in FIGS. 1-3, one or more of the sensors 34, when placed in the breathing circuit 26, may also provide feedback signals back to the electronic control circuitry 44 of the ventilator 16 via a feedback loop 52, as shown in FIG. 3. More specifically, a signal in the feedback loop 52 may be proportional, for example, to gas flows and/or airway pressures in the patient branch 42 leading to the lungs 30 of the patient 12. Inhaled and exhaled gas concentrations (e.g., oxygen (O₂), carbon dioxide (CO₂), nitrous oxide (N₂O), and inhalation anesthetic agents), flow rates (including, for example, spirometry), and gas pressurization levels, etc., may be captured by the sensors 34, as well as durations of time periods between when the ventilator 16 permits the patient 12 to inhale and exhale, and when the patient's natural inspiratory and expiratory flows cease.

Thus, the electronic control circuitry 44 of the ventilator 16 may also control displaying numerical and/or graphical information from the breathing circuit 26 on the monitor 38 of the medical system 10 (as shown in FIGS. 1-2), as well as other patient 12 and/or system 10 parameters from other sensors 34 and/or the processing terminal 36 (as shown in FIG. 1). In other embodiments, various components can also be integrated and/or separated, as needed and/or desired.

The electronic control circuitry 44 of FIG. 3 may also coordinate and/or control, for example, elements depicted in FIG. 2, including other ventilator setting signals 54, ventilator control signals 56, and/or a processing subsystem 58 for receiving and processing signals from the sensors 34. The electronic control circuitry 44 may further be configured to display signals for the monitor 38 and/or the like, control alarms 60, and/or an operator interface 62, which may include a graphical user interface (GUI) displayed at the monitor 38, and one or more input devices 64, etc., all as demanded and/or desired and interconnected suitably.

The processing subsystem 58 may be located at the processing terminal 36 of FIG. 1 and may include various electronic components, e.g., hardware, for receiving and transmitting signals, and processing thereof. For example, the processing subsystem 58 may include a controller (e.g., a processor) configured to receive signals from the sensors 34, which may include pressure sensors, flow sensors, sensors monitoring statuses of valves and switches, etc., and send control signals to actuators of the medical system, such as pressure regulators, the valves and switches, etc., in response to the sensor signals. The controller may be a microcomputer, including a microprocessor unit, input/output ports, an electronic storage medium (including nontransitory memory) for storing executable programs and parameter setting values. The controller may be programmed with computer readable data representing instructions executable to perform the methods described herein as well as other variants that are anticipated but not specifically listed.

The components of FIGS. 1-3 are depicted for illustration, wherein various other components may also be integrated and/or separated therein, based on demand and/or as desired. Other components, for example, one or more power supplies for the medical system 10 and/or anesthesia machine 14 and/or ventilator 16, etc. (not shown) may be provided.

As described above, the ventilation system may be operated in the mechanical mode or the manual mode. When operating in the mechanical mode, PEEP may be monitored and provided to maintain a positive pressure in the patient's airways at an end of an expiration phase, thereby mitigating collapse of the airways after exhalation. The ventilation system may be adjusted to the manual ventilation mode under circumstances such as initial intubation and during weaning procedures. When the patient is ventilated using a manual circuit including a bag, oxygen (and other gases) may be provided to the patient's airways by compressing the bag. As the bag is compressed and refilled with breathing gases (e.g., fresh gas) repeatedly, pressure inside the bag may increase until the pressure reaches a maximum pressure (Pₘₐₓ) that is set based on a pressure control system of the manual circuit, which may include an adjustable pressure limiting (APL) valve.

While the APL valve allows control over a maximum pressure of the bag, pressure in the patient's airways during expiration may not be regulated via the bag. Furthermore, when the operating mode of the ventilation is adjusted from the manual ventilation mode to the mechanical ventilation mode, a pressure of gas delivery (e.g., fresh gas or ventilator bias flow) to the patient may be increased to the APL valve pressure setting. The increased gas pressure may lead to barotrauma or volutrauma.

In one example, the issues above may be at least partially addressed by configuring a ventilation system with a manual circuit that enables PEEP during operation of the ventilation system in a manual ventilation mode, e.g., a manual ventilation PEEP delivery system. The manual ventilation PEEP delivery system may include discharging of an additional volume of gas (e.g., breathing gas) directly to the scavenging circuit during expiration to allow a target level of expiratory gas pressure to be attained. The additional volume may be discharged during inspiration to constrain a Pₘₐₓ to a desired level.

Implementation of the manual ventilation PEEP delivery system may leverage existing infrastructure for controlling PEEP in the ventilation system. To enable PEEP delivery during manual ventilation, an additional device, such as a pressure regulating valve (PRV), may be arranged between the manual resuscitator and the scavenging circuit.

In addition, a three-port solenoid valve may channel the flow of a pilot gas (e.g., air and/or oxygen) used for mechanical ventilation to the PRV during manual ventilation. The pilot gas may provide a pilot pressure that controls a status of the PRV. Parameters for PEEP and Pₘₐₓ may be set by the user and fed to the algorithms implemented at and executed by a controller of the ventilation system. As such, a PEEP pressure setting used for manual ventilation may be applied to a PEEP pressure setting during mechanical ventilation when the ventilation system is adjusted from the manual ventilation mode to the mechanical ventilation mode.

In one example, the manual ventilation PEEP delivery system may be retrofit to an already existing ventilation system. A block diagram representing an architecture 400 of a manual ventilation system (also, a manual ventilation circuit) incorporating the manual ventilation PEEP delivery system is depicted in FIG. 4. The manual ventilation system may be included in, for example, a medical system such as the medical system 10 of FIGS. 1-3 for providing manually controlled respiration to a patient. Conventional gas paths (e.g., existing), connecting already existing components of the manual ventilation system are indicated in solid lines while new gas paths, as well as new components, corresponding to the manual ventilation PEEP delivery system are shown in dashed lines.

The conventional gas paths may include gas flow between a bag 402 and a patient via an inspiration/expiration port 404 of the bag 402. Fresh gas (e.g., breathing gas) from a gas source 406 may be delivered to the patient via manipulation of the bag 402, which may be filled with the fresh gas at a start of an inspiration cycle. A pilot gas (e.g., air and/or oxygen) may also flow from the bag 402 to a PRV 408 for providing PEEP during operation of the ventilation system in a manual ventilation mode. In one example, the PRV 408 may be a modified exhalation valve where gas flow therethrough may be controlled by a solenoid and a diaphragm. A new gas path may be provided by adapting the bag 402 with a T-junction 409, that couples the bag 402 to the patient by the inspiration/expiration port 404 and to a scavenging circuit 410 of the medical system via the PRV 408.

The PRV 408 may be controlled by a pilot pressure delivered by a vent engine 412, as described further below, with reference to FIG. 5, and may be configured as an electronically activated, pneumatically driven diaphragm valve that balances a pressure in the bag 402 against the pilot pressure. For example, the fresh gas may flow from the bag 402 to the scavenging circuit 410 when the bag pressure is greater than the pilot pressure, where the bag pressure includes a pressure of gas paths fluidically coupling the bag 402 to other components of the manual ventilation PEEP delivery system. When the bag pressure is less than the pilot pressure, the gas path thereto may be blocked. The pilot pressure may provide a flow of the pilot gas that maintains PEEP when providing respiration to the patient using the bag and may be controlled according to software algorithms based on monitoring of the inspiration/expiration gas flow and corresponding pressure waveforms (e.g., P_{AW}), as provided by sensor data. Further details of implementation of the manual ventilation PEEP delivery system are illustrated in FIG. 5.

Turning now to FIG. 5, a portion of a pneumatic system 500 of a ventilation system is shown as a schematic diagram. The ventilation system may be configured with a manual ventilation PEEP delivery system 501. Gas paths are indicated by lines, including triangles indicating directions of dedicated gas flow. The directions of dedicated gas flow may represent gas in gas lines that consistently flows in a direction indicated by a respective triangle. At least some components shown in FIG. 5 are in common with the medical system 10 of FIGS. 1-3 and with the architecture 400 of the manual ventilation PEEP delivery system of FIG. 4, and may therefore operate in a similar manner. The pneumatic system 500 may be an embodiment of the pneumatic circuitry 46 of FIG. 3 and includes a bag 502 for providing manual ventilation.

The bag 502 may an example of the manual resuscitator 28 illustrated in FIG. 1. For example, the bag 502 may be coupled to a manifold with various ports and connectors. Further, the bag 502 may be connected to a gas source, which may be a gas mixer 518 of the ventilation system, by a first path of fresh gas flow 550 (where fresh gas refers to air, oxygen, nitrous oxide, and/or anesthetic agents) that extends from the gas mixer 518 to the bag 502 via a BTV switch 504. A continuous flow of fresh gas may be delivered to the bag 502 via the first path of fresh gas flow 550 during manual ventilation, regardless of breathing phase.

In addition, a second path of fresh gas flow 560 may extend between the gas mixer 518 and the bag 502 which may allow the bag 502 to be filled upon commencing manual ventilation. The fresh gas flow through the second path of fresh gas flow 560 may be enabled by activating a switch 562, e.g., an “O₂ flush” switch, which may be connected in series to the second path of fresh gas flow 560. By activing the switch 562, the bag 502 may be filled to a target PEEP pressure at a start of manual ventilation and the fresh gas in the bag 502 may be delivered to a patient by manual compression of the bag 502.

For example, when the bag 502 is compressed during an inhalation phase, a pressure in the manual ventilation PEEP delivery system 501 may increase to a pre-set Pₘₐₓ value and excess pressure may be released through, for example, an APL valve 510, or to a scavenging circuit through a PRV 534, described further below. During an exhalation phase, continuous flow of fresh gas from the first path of fresh gas flow 550 may partially fill the bag. The continuous flow of fresh gas from the first path of fresh gas flow 550 may increase a pressure of the bag 502 above a PEEP setting when an end of the exhalation phase approaches, where the PEEP setting may be set at the beginning of the previous inhalation phase. Excess pressure above the PEEP setting may be vented through the PRV 534.

The APL valve 510 may be included in the manual ventilation PEEP delivery system 501 and coupled to the bag 502. The APL valve 510 may control the Pₘₐₓ of the bag 502 when manual ventilation is executed while the ventilation system is not powered. For example, the APL valve 510 may operate as a back-up, manual (e.g., non-electronic) method for controlling the Pₘₐₓ. During operation of the ventilation system in the manual ventilation mode while electrically powered, the APL valve 510 may remain in a closed, inactive status when manually set to a maximum pressure. The manual pressure setting of the APL valve 510 may impose a maximum allowed pressure of the bag irrespective of an electronic Pₘₐₓ setting of the ventilation system, and may allow excess gas from the bag 502 to be flowed to the scavenging circuit via a first fresh gas path 503 of the manual ventilation PEEP delivery system 501.

Operation of the ventilation system may be toggled between manual ventilation and mechanical ventilation by adjusting the BTV switch 504, which may be analogous to the BTV 32 of FIG. 1. As an example, the BTV switch 504 is shown in a first position for operation in a manual ventilation mode where the bag 502 is fluidically coupled to a patient circuit (e.g., a breathing system), the patient circuit including devices for delivering gases to a patient's lungs. For example, the patient circuit may include a CO₂ absorber, a pressure relief valve, check valves, flow sensors, pressure sensors, a Y-connector, a limb coupling the Y-connector to a patient, oxygen sensors, etc.

When the BTV switch 504 is adjusted to a second position for operation in a mechanical ventilation mode, the patient circuit may instead be fluidically coupled to a vent engine 506 via a reciprocating unit 508. The reciprocating unit 508 may be a long gas flow channel and may include a bellows and a bottle. The reciprocating unit 508 may be driven by the vent engine 506 and fluidically coupled to an exhalation valve 512 for mechanical ventilation, in addition to other circuits and/or components. For example, exhaled gases from the patient may flow through the BTV switch 504 (when the BTV is in the second position) into the reciprocating unit 508. From the reciprocating unit 508, the exhaled gases may flow through an exhalation line 505, as well as a passage including a pop-off valve 514, to an exhalation valve 512. From the exhalation valve 512, the exhaled gas may be sent to the scavenging circuit.

The vent engine 506 includes a free breathing valve 516, which may be a redundant mechanical valve that operates without reliance on electrical power. The free breathing valve 516 may allow the patient to inhale between inspiration cycles of mechanical ventilation, therefore enabling spontaneous breathing. Atmospheric air may be drawn into the vent engine 506 and delivered to the patient through the reciprocating unit 508 during spontaneous breathing.

During inspiration, fresh gas may be provided from the gas mixer 518 along the first path of fresh gas flow 550 to the reciprocating unit 508 or to the bag 502 depending on the position of the BTV switch 504. For example, when the BTV switch 504 is in the first position, the fresh gas may be diverted to the bag 502 through a manual breathing gas line 552, via the BTV switch 504. The fresh gas may also flow into a second fresh gas path 507 of the manual ventilation PEEP delivery system 501 along which the PRV 534 is arranged. A pressure in the second fresh gas path 507 may be equal to a pressure in the manual breathing gas line 552 and the bag 502, as well as a pressure in the second path of fresh gas flow 560. When the pressure in the second fresh gas path 507 exceeds a Pₘₐₓ setting of the ventilation system during manual ventilation, excess fresh gas may be vented to the scavenging circuit through a scavenging line 509.

When the BTV switch 504 is in the second position, the fresh gas may instead be diverted to the reciprocating unit 508 and the exhalation valve 512. The gas mixer 518 may include various filters, flow controllers, pressure transducers, pressure regulators, valves, and flow measurement devices for controlling mixing and delivery of the oxygen, air, nitrous oxide, and/or anesthetic agents to the patient circuit.

A drive gas and a pilot gas, the drive gas and the pilot gas being oxygen and/or air, may be also delivered to the reciprocating unit 508 and the exhalation valve 512 during mechanical ventilation and to the PRV 534 during manual ventilation. The drive gas and the pilot gas may be passed through a filter 520 arranged between a gas source (such as an oxygen gas source) and a Drive and PEEP gas control unit 522. The oxygen gas source may be a pipeline or cylinder. For example, the drive gas may be provided to the reciprocating unit 508 and the exhalation valve 512 from the gas source to provide a driving force for the reciprocating unit 508. The drive gas may reach the exhalation valve 512 via a three-port, two-way solenoid (TPTW) valve 532 which may be an electronically-actuated solenoid valve that toggles between two positions to either channel the drive gas to the exhalation valve 512 or the pilot gas to the PRV 534.

When the BTV switch 504 is switched to the first position to adjust operation of the ventilation system to the manual ventilation mode, the pilot gas is delivered to the PRV 534 from the gas mixer 518 via the TPTW valve 532, with a position of the TPTW valve 532 adjusted accordingly. The pilot gas provides a pilot pressure in the manual ventilation PEEP delivery system 501 that provides and controls PEEP during manual ventilation.

The Drive and PEEP gas control unit 522, along with its electronic control, may be used to determine and control an amount of drive gas to be delivered to the reciprocating unit 508 and an amount of pilot gas to be delivered to the TPTW valve 532 based on a ventilation mode of the ventilation system (e.g., manual versus mechanical), as well as parameters that may be set by an operator, such as a clinician. Further, the Drive and PEEP gas control unit 522 may control a timing at which the pilot gas flow is diverted to the reciprocating unit 508 and the exhalation valve, or to the PRV 534.

The Drive and PEEP gas control unit 522 may include various devices for controlling gas flow to provide gas for PEEP during mechanical ventilation. For example, the Drive and PEEP gas control unit 522 may be configured with (but not limited to) one or more of flow control devices, pressure regulators, valves, etc. As an example, the Drive and PEEP gas control unit 522 may include a solenoid valve that adjusts the unit between flowing the drive gas to the reciprocating unit 508 and the exhalation valve 512 or flowing the pilot gas to the PRV 534.

For example, at a junction point 524 at which the Drive and PEEP gas control unit 522 is located, at least a portion of the drive gas from the gas source may be directed through the Drive and PEEP gas control unit 522 during mechanical ventilation to flow to the reciprocating unit 508, as described above. An over pressure relief valve 528 may be included in a path between the junction point 524 and the reciprocating unit 508. At least a portion of the drive gas from the gas source may concurrently be diverted at the junction point 524 through the TPTW valve 532 to the exhalation valve 512 via a drive gas branch 535 extending between the TPTW valve 532, with a bleed flow resistor 515 to vent gas to ambient surroundings, and the exhalation valve 512.

During manual ventilation, the pilot gas may be directed from the gas source, through the TPTW valve 532 to the PRV 534 by the Drive and PEEP gas control unit 522. A PEEP control gas path 531 extending between the junction point 524 and the TPTW valve 532 may include a passage with the bleed flow resistor 515 to vent gas from the PEEP control gas path 531 to ambient surroundings, thereby regulating pressure in the PEEP control gas path 531. A pilot gas branch 533 may extend between the TPTW valve 532 and the PRV 534.

The TPTW valve 532 may be adjusted by varying a position of a solenoid of the TPTW valve 532 to fluidically couple the Drive and PEEP gas control unit 522 to either the exhalation valve 512, or to the PRV 534, depending on the position of the TPTW valve 532 (e.g., the position of the solenoid of the TPTW valve 532). For example, the TPTW valve 532 may include a first port that is an inlet for receiving the drive gas or the pilot gas from the Drive and PEEP gas control 522 (through components arranged therebetween), a second port that is an outlet for flowing the drive gas to the exhalation valve 512, and a third port that is an outlet for flowing the pilot gas to the PRV 534. The TPTW valve 532 may be toggled between fluidically coupling the first port to the second port, and thereby decoupling the first port from the third port, or fluidically coupling the first port to the third port and thereby decoupling the first port from the second port.

A gas destination selected based on the TPTW valve 532 state may therefore be adjusted based on the solenoid of the TPTW valve 532. In one example, the solenoid may be magnetically actuated to slide such that energization of an electromagnet compels positioning of the solenoid to fluidically couple the first port to the third port while de-energization of the electromagnet shifts the solenoid to fluidically couple the first port to the second port. By demanding energization for the solenoid to fluidically couple the first port to the third port, the bag 502 may be used for manual resuscitation even during loss of power to the ventilation system.

In one example, the state of the TPTW valve 532 may be adjusted according to the position of the BTV switch 504. For example, when the BTV switch 504 is in the first position for operation in the manual ventilation mode, the TPTW valve 532 may be adjusted to allow the pilot gas to flow from the gas mixer 518, through the Drive and PEEP gas control unit 522, and to the PRV 534. The pilot pressure delivered to the TPTW valve 532 may be controlled by the Drive and PEEP gas control unit 522, such as by the solenoid valve of the Drive and PEEP gas control unit 522, as an example.

For example, the TPTW valve 532 may configured as a selector switch having the two positions described above, to toggle the pilot gas flow out of the second port or the third port of the valve. The pilot gas flow from the Drive and PEEP gas control unit 522 may be increased to accommodate a higher pilot pressure demand or decreased accordingly when the pilot pressure demand is reduced. In one example, the pilot pressure demanded of the Drive and PEEP gas control unit 522 may be determined based on parameters entered by the operator at a user interface of the ventilation system, such as the GUI of the monitor 38 of FIG. 1, where the parameters include a PEEP and a Pₘₐₓ value. The bleed flow resistor 515 may provide controlled venting of the pilot gas to ambient atmosphere to regulate the pilot pressure in the PEEP control gas path 531 and reset a pressure in the PEEP control gas path 531 after each breath cycle.

The PRV 534 may incorporate each of a solenoid and a diaphragm to control a pressure in the bag 502, and therefore in airways of the patient, during operation in the manual ventilation mode. A position of the solenoid may be controlled by an electromagnet, which toggles the solenoid between two positions depending on whether the electromagnet is energized or de-energized. The PRV 534 may be fluidically coupled to the bag 502, the scavenging circuit, and the breathing system (e.g., the patient circuit) through a first portion of the PRV 534 and fluidically coupled to the Drive and PEEP gas control unit 522 (via the TPTW valve 532) through a second portion of the PRV 534. The first and second portions of the PRV 534 may be divided by the diaphragm. Further details of the PRV 534 are shown in FIGS. 6A-6C.

Turning now to FIGS. 6A-6C, the PRV 534 is depicted in a first configuration 600 in FIG. 6A, corresponding to a de-energized state (with respect to the electromagnet controlling a position of a solenoid 602), in a second configuration 620 in FIG. 6B, corresponding to an energized state during manually ventilated inspiration, and in a third configuration 640 in FIG. 6C, corresponding to an energized state during manually ventilated expiration. As described above, the PRV 534 has a first portion 630 fluidically coupled to fresh gas passages of the manual ventilation PEEP delivery circuit the bag (e.g., the manual ventilation PEEP delivery system 501 and the bag 502 of FIG. 5), and the breathing system (e.g., the patient circuit of FIG. 5), and a second portion 632 fluidically coupled to the Drive and PEEP gas control unit (e.g., the Drive and PEEP gas control unit 522 of FIG. 5). Orientations of the bag (e.g., the bag 502 of FIG. 5), the scavenging circuit, and the TPTW valve (e.g., the TPTW valve 532 of FIG. 5) relative to the PRV 534 are indicated, where dashed arrows represent positioning of the bag and the scavenging circuit according to gas flow direction. Shaded block arrows indicate actual gas flow, as described further below.

In the first configuration 600 of FIG. 6A, the solenoid 602 may be in contact with and press against a first face 603 of a diaphragm 604 of the PRV 534 due to a spring force of a solenoid spring 606 coupled to the solenoid 602. The ventilation system may be in the mechanical ventilation mode in FIG. 6A with the BTV switch (e.g., the BTV switch 504 of FIG. 5) in the second position and with the electromagnet of the solenoid 602 de-energized. When in the first configuration 600, fresh gas in the manual ventilation circuit does not flow through the PRV 534 to the scavenging circuit. Instead, a path to the scavenging circuit (e.g., the scavenging line 509 of FIG. 5) is blocked by a position of the diaphragm 604. The path to the scavenging circuit is closed as a result of the solenoid 602 pressing against the diaphragm 604 and inhibiting deformation and/or displacement of the diaphragm 604.

In the second configuration 620 of FIG. 6B, the BTV switch is adjusted to the first position to place the ventilation system in the manual ventilation mode. The electromagnet may be energized in response, causing the solenoid 602 to retract and become spaced away from the diaphragm 604, against the spring force of the solenoid spring 606. In other words, the solenoid 602 no longer presses against the first face 603 of the diaphragm 604. A respiratory cycle of the patient may be undergoing inspiration as the bag is compressed, as determined based on signals from one or more pressure sensors of the patient circuit.

The pilot gas may be delivered to the PRV 534 at a pilot pressure determined by the Drive and PEEP gas control unit, as indicated by arrow 608, according to a pressure setting for the Pₘₐₓ. For example, the pilot pressure may be equal to the Pₘₐₓ setting (e.g., as set and input by the operator) which may also determine a pressure provided by the flow of the fresh gas through the first portion 630 of the PRV 534. The pilot pressure may thereby control the Pₘₐₓ in the bag, which is transmitted as a pressure to the patient's airways (e.g., P_{aw}) in a manner analogous to use of the APL valve. If the pressure in the bag exceeds the Pₘₐₓ setting, the diaphragm may be displaced or depressed, as shown in FIG. 6C, to vent excess fresh gas to the scavenging circuit. Flow of the fresh gas from the bag (or from the breathing system) is indicated by arrow 610.

For example, although the solenoid 602 is not exerting a force against the first face 603 of the diaphragm 604, the pilot gas flow provided by the Drive and PEEP gas control unit may instead exert pressure against the first face 603 of the diaphragm 604. The exerted pressure may correspond to the Pₘₐₓ setting, which may exert a force against the first face 603 of the diaphragm 604. During inspiration, when a pressure in the bag exceeds the Pₘₐₓ, a pressure exceeding that of the Pₘₐₓ communicated from the bag (such as during filling of the bag with gas) may exert a force against a second face 605 of the diaphragm 604, opposite of the force exerted by the pilot pressure on the first face 603. The greater pressure on a bag side of the diaphragm 604, e.g., against the second face 605, may cause the diaphragm to be displaced away from its original position, towards the solenoid 602, as illustrated in FIG. 6C. The displacement may allow excess pressure, relative to the Pₘₐₓ, to be vented to the scavenging circuit. When the excess pressure is dissipated, the diaphragm 604 may return to its original position as shown in FIG. 6B, again blocking flow between the bag and the scavenging circuit.

In the third configuration 640 of FIG. 6C, the ventilation system is operating in the manual ventilation mode during an expiration cycle, as determined based on the signals from the pressure sensors of the patient circuit. The electromagnet of the solenoid 602 is energized and the solenoid 602 is retracted similarly as described above. In response to adjustment of operation to the expiration cycle, the pilot pressure is modified to correspond to a PEEP setting selected and input by the operator. For example, the PEEP setting may be a lower pressure than the Pₘₐₓ setting. A pressure against the first face 603 of the diaphragm 604 in the third configuration 640 is therefore less than the pressure against the first face 603 in the second configuration 620.

At a start of the expiration cycle, the lower pressure of the PEEP setting relative to the Pₘₐₓ setting causes the pressure in at the bag side of the diaphragm 604 to be greater than the pilot pressure provided by the Drive and PEEP gas control unit when operation initially switches from the inspiration cycle to the expiration cycle. The excess pressure on the bag side of the diaphragm 604 may displace the diaphragm towards the solenoid 602, by a distance 642, allowing the gas from the bag to be vented to the scavenging circuit, as indicated by arrow 644. The diaphragm 604 may remain displaced until the pressure on the bag side is equal to the PEEP setting. Upon pressure equilibration across the diaphragm 604, the diaphragm 604 may shift back to its original position (e.g., as shown in FIGS. 6B), thereby blocking gas flow between the bag and the scavenging circuit.

By leveraging a gas flow infrastructure used for mechanical ventilation, control of PEEP and Pₘₐₓ may be provided during manual ventilation. Precise electronic pressure control, based on operator-selected settings, may be enabled by a manual ventilation PEEP delivery system as described herein. The pressure control may be maintained across both mechanical and manual ventilation, which may circumvent derecruitment of a patient's lungs and reduce a likelihood of barotrauma and volutrauma to the patient. The manual ventilation PEEP delivery system (e.g., the manual ventilation PEEP delivery system 501 of FIG. 5) may be incorporated into already existing ventilation configurations and may retain an ability to execute manual resuscitation, e.g., ventilation via a bag without PEEP, in events of power loss. Enabling PEEP and Pₘₐₓ control during manual ventilation may preclude dependency on operator experience which may lead to suboptimal and inconsistent pressure control. For example, during manual resuscitation relying on an APL valve for pressure control of the bag, the operator may determine suitable pressure by based on feel (e.g., by squeezing the bag and evaluating a resistance of the bag to compression) and manipulate the APL valve to Pₘₐₓ according to the operator's estimate of a suitable APL valve adjustment. The operator may not be able to reset the bag to a suitable baseline pressure and cannot deliver PEEP.

The manual ventilation PEEP delivery system may incorporate a TPTW valve, a pneumatically-controlled PRV, and a pathway between the PRV and a scavenging circuit to allow PEEP to be maintained when a ventilation system is adjusted to a manual ventilation mode. At a start of each expiration cycle, the pilot pressure may be reset to a target PEEP value as the baseline pressure. A gradual accumulation of pressure and corresponding increase in baseline pressure of the ventilation system is thereby circumvented. In one example, the operator may input a target value for each of the PEEP and the Pₘₐₓ at an operator interface of the ventilation system, such as the operator interface 62 of FIG. 1. The PEEP, Pₘₐₓ, and corresponding feedback mechanisms, may be continuously monitored and displayed in real-time at a display screen, such as the monitor 38 of FIGS. 1 and 2. The operator may observe the current PEEP value and correlate the value with other hemodynamic parameters and waveforms to assess relationships between data in an efficient and meaningful manner.

A processor of the ventilation system may be configured with software algorithms for controlling PEEP and Pₘₐₓ during manual ventilation, in addition to mechanical ventilation. When the ventilation system is adjusted to the manual ventilation mode, a corresponding signal may be sent to the processor. In response to the signal, the processor may command the TPTW valve (e.g., by energizing an electro-magnet of a solenoid) to divert gas flow from a vent engine of the ventilation system to the manual ventilation PEEP delivery system. The gas flow may be regulated to provide a pressure in the bag and gas lines of the manual ventilation PEEP delivery system according to the target value input by the operator. Upon adjustment of operation to the mechanical ventilation mode, as triggered by the BTV switch, the PEEP setting, as well as the Pₘₐₓ setting, may be maintained.

During operation in the manual ventilation mode, the software algorithms may include instructions for detecting inhalation (e.g., inspiration) and exhalation (e.g., expiration) phases of respiration based on pressure waveform and flow data obtained from pressure and mass flow sensors of the ventilation system. At a beginning of an inspiration cycle, a pilot pressure provided by the Drive and PEEP gas control unit may be adjusted to the Pₘₐₓ value set by the operator and at a beginning of an expiration cycle, the pilot pressure may be modified to the PEEP value set by the operator. When breaths of the patient are not detected, e.g., the bag is not being compressed or filled with gas, the software algorithms may include instructions to automatically set the pilot pressure to the PEEP value to ensure that the patient's airways are maintained with PEEP. In addition, the software algorithms may also enable adjustment and correction of the pilot pressure delivered by the Drive and PEEP gas control unit breath by breath, in real-time, to achieve the set PEEP based on feedback from pressure sensors of the manual ventilation PEEP delivery system. As a result, the pressure in the patient's airways may be continuously optimized according to respiratory phase. Variations in the patient's condition, which may affect respiration and oxygenation, may thereby be accounted for during both manual ventilation and mechanical ventilation. Further, seamless transition between manual and mechanical ventilation is enabled, e.g., without disruption or abrupt changes to Pₘₐₓ and PEEP.

A method 700 for operating a ventilation system equipped to operate in both a mechanical and a manual ventilation mode is depicted in FIG. 7 and a method 800 for providing PEEP and Pₘₐₓ during operation of the ventilation system in the manual ventilation mode, e.g., a bag mode, is shown in FIG. 8. For example, methods 700 and 800 may be continuously executed during operation to enable smooth transitioning of ventilation between the manual and mechanical ventilation modes. The ventilation system may include a controller, having a microprocessor as described above, configured with instructions stored on a memory of the controller for executing methods 700 and 800. The instructions may be executed in conjunction with signals received from sensors of the ventilation system, such as the sensors described above with reference to FIGS. 1-3. The controller may employ actuators of the ventilation system, such as valves, switches, etc., to adjust operation of the ventilation system, as described above. In one example, the ventilation system may be the medical system 10 of FIGS. 1-3, which may include a manual circuit, such as the manual ventilation PEEP delivery system 501 of FIG. 5 with the architecture 400 of FIG. 4, as well as a mechanical circuit, such as the vent engine 506 of FIG. 5.

At 702, method 700 includes confirming if the ventilation system is operating in the manual ventilation mode or the mechanical ventilation mode. The ventilation system may be activated, e.g., powered and operating, and providing ventilation to a patient. The operating mode of the ventilation may be determined based on a position of a BTV switch, such as the BTV switch 504 of FIG. 5. For example, an operator may manually adjust the position of the BTV switch.

If the ventilation system is operating in the mechanical ventilation mode, method 700 includes adjusting a TPTW valve at 704, such as the TPTW valve 532 of FIG. 5, to direct a flow of a pilot gas from a gas source to an exhalation valve of a mechanical ventilation circuit of the ventilation system. A rate of the pilot gas flow may be controlled by a gas control unit, such as the Drive and PEEP gas control unit 522 of FIG. 5. For example, directing the pilot gas flow to the mechanical ventilation circuit may include de-energizing the TPTW valve if the ventilation system is adjusted from the manual ventilation mode to the mechanical ventilation mode, or maintaining the TPTW de-energized valve if the ventilation system is already operating in the mechanical ventilation mode. As such, the de-energized position may represent a normal or default position of the TPTW valve when the TPTW valve is not actuated. The pilot gas only flows to the mechanical circuit through the TPTW valve and the pilot gas flow to the manual circuit is therefore blocked. Values for PEEP and Pₘₐₓ may be applied at 706 according to software algorithms stored in the controller's memory and used during mechanical ventilation. The method returns to the start.

In one example, the software algorithms for providing the PEEP and Pₘₐₓ values to be used during mechanical ventilation may include instructions for retrieving the PEEP and Pₘₐₓ values used during an expiration cycle of a previous manual ventilation event. For example, the previous manual ventilation event may include a most recent operation of the ventilation system in the manual ventilation mode. As an example, the patient may be manually ventilated during intubation and/or weaning. Upon completing intubation, ventilation of the patient may be adjusted to the mechanical ventilation mode and the PEEP and Pₘₐₓ values used during operation in the manual ventilation mode may be transferred for use during operation in the mechanical ventilation to maintain consistency in the patient's P_{aw}.

Alternatively, if the ventilation system is operating in the manual ventilation mode, method 700 proceeds to 708 to adjust the TPTW valve to direct a pilot gas flow to the manual circuit. For example, the TPTW valve may be energized, if the ventilation system is adjusted from the mechanical ventilation mode to the manual ventilation mode, or may remain energized if the ventilation system is already in the manual ventilation mode. The pilot gas is channeled exclusively to the manual circuit from the gas source and the gas control unit, through the TPTW valve. The flow rate of the pilot gas, as described above, may be adjusted and controlled by the gas control unit. Further, at 710, a PEEP value and a Pₘₐₓ value input by the operator may be applied to regulate gas flow through the TPTW valve to the manual circuit, details of which are provided in FIG. 8 and described further below. In one example, the PEEP and Pₘₐₓ values may be input by the operator at a GUI of the ventilation system and received by the controller to be used as target settings at the gas control unit. The method returns to the start.

Turning now to FIG. 8, at 802, method 800 includes activating the manual circuit of the ventilation system. For example, activating the manual circuit may include adjustment of the ventilation system from mechanical ventilation to manual ventilation. A transition from mechanical to manual ventilation may be timed at an end of a respiration cycle, before a start of a subsequent cycle. Activating the manual circuit may also include confirming and applying the PEEP and Pₘₐₓ values input by the user at 804. In other words, the input values may be used as target values for determining settings for actuators of the manual circuit, as well as for a pilot gas source, such as the gas mixer 518 of FIG. 5. Activating the manual circuit may further include activating the TPTW valve and a PRV (such as the PRV 534 of FIG. 5) at 806 by energizing respective electromagnets of the valves. By energizing the respective electromagnets, the TPTW valve may direct a desired flow rate of gas (e.g., the pilot gas) to the manual circuit from the gas control unit corresponding to the set PEEP and Pₘₐₓ values. The PRV may vent pressure in excess of the target pressure values to the scavenging circuit. Additionally, at 808, activating the manual circuit may include obtaining and monitoring flow and P_{aw} data from sensors of the manual circuit as well as from sensors of the gas source.

At 810, method 800 includes determining if a breath is detected to confirm if manual ventilation is currently in an expiration or inspiration phase. For example, the flow and P_{aw} data may be analyzed in real-time to identify a current respiration status of the ventilation system (and of the patient). If the breath is not detected, e.g., neither inspiration or expiration is detected at the patient, ventilation may be temporarily paused and method 800 returns to 802 to set the pilot pressure according to the target PEEP value. PEEP is thereby maintained in the patient's lungs, mitigating collapse of the patient's airways.

If the breath is detected, method 800 continues to 812 to confirm if a current ventilation cycle is an expiration phase. If the ventilation cycle is not in the expiration phase, the ventilation cycle is in an inspiration phase. Method 800 proceeds to 814 to adjust gas flow through the TPTW valve to deliver the pilot pressure to the PRV at the start of the inspiration phase, according to the input Pₘₐₓ value. For example, the bag may be inflated to the Pₘₐₓ value such that the pressure transferred to the patient's airways upon compression of the bag does not exceed the Pₘₐₓ value. Pressure in excess of the Pₘₐₓ value in the bag may be vented to the scavenging circuit through the PRV by displacement of a diaphragm of the PRV. Method 800 then continues to 818, as described further below.

If, however, the ventilation cycle is confirmed to be in the expiration phase, method 800 proceeds to 816 to adjust gas flow from the gas control unit (e.g., the Drive and PEEP gas control unit) to deliver the pilot pressure to the PRV according to the input PEEP value. Any pressure in the bag in excess of the PEEP value may be vented to the scavenging circuit. As the patient exhales passively, exhaled gas may be flowed to the scavenging circuit, increasing a pressure of an exhalation portion of the manual circuit. Any pressure in the manual circuit in excess of the PEEP value may be vented to the scavenging circuit by displacement of the PRV diaphragm of the PRV until the pressure in the manual circuit is equal to the PEEP value. Upon equilibration of pressure on either side of the diaphragm, the diaphragm is returned to an original position, blocking flow of gas out of the manual circuit and retaining a pressure in the manual circuit equal to the PEEP value. Furthermore, the Drive and PEEP gas control unit may be adjusted at a start of a subsequent inspiration phase to deliver the pilot pressure corresponding to the Pₘₐₓ value, as described above.

At 818, method 800 includes determining if the P_{aw} is equal to the PEEP value at a start of a next (e.g., subsequent) expiration phase. If the P_{aw} is equal to the PEEP value, method 800 returns to 802 to maintain the manual circuit active and continue monitoring and providing the pilot pressure for PEEP and Pₘₐₓ control. If the P_{aw} does not equal the PEEP value, method 800 continues to 820 to determine if the P_{aw} is less than the PEEP value. If the P_{aw} is greater than the PEEP value, the pilot pressure provided by the Drive and PEEP gas control unit is decreased at 822 by adjusting the Drive and PEEP gas control unit to deliver the gas to the PRV at a lower flow rate. Method 800 then returns to the start. Alternatively, if the P_{aw} is less than the PEEP at 820, method 800 proceeds to 824 to increase the pilot pressure provided by the Drive and PEEP gas control unit by increasing the flow rate. Method 800 then returns to the start.

Exemplary variations in operating parameters during operation of a ventilation system having a manual ventilation PEEP delivery system, such as the manual ventilation PEEP delivery system 501 of FIG. 5 are shown in FIG. 9 in a graph 900. Graph 900 includes a first plot 902 representing P_{aw} of a patient receiving ventilation, a second plot 904 representing a status of a PRV, a third plot 906 representing a rate of fresh gas flow from the PRV to a scavenging circuit of the ventilation system, and a fourth plot 908 representing a pressure in a bag of the ventilation system used to deliver gas to a patient's airways. Time is shown along the x-axis, with events of significance indicated, and variables along the y-axes vary depending on the parameter. For example, P_{aw} varies continuously between a set Pₘₐₓ value and a set PEEP value along the y-axis of the first plot 902, the status of the PRV varies between open and closed along the y-axis of the second plot 904, which corresponds to displacement of a diaphragm of the PRV from its original, resting position. For the third plot 906, gas flow increases upwards along the y-axis, and bag pressure also varies between the Pₘₐₓ and the PEEP for the fourth plot 908.

At t0, an expiration cycle begins. The patient's P_{aw} is at the Pₘₐₓ value due to completion of a previous inspiration cycle, the PRV is closed (e.g., the diaphragm is not displaced and flow between the bag and the scavenging circuit is blocked) with a pilot pressure set at the PEEP value, and the bag pressure is also at the Pₘₐₓ value. Between t0 and t1, the P_{aw} and the bag pressure decrease rapidly as the patient exhales and a pressure setting of the manual ventilation PEEP delivery circuit is adjusted to the PEEP value from the Pₘₐₓ value. The PRV initially becomes more open due to a rise in pressure in an exhalation portion of manual breathing circuit of the manual ventilation PEEP delivery system, and becomes less open after the initial rise. The gas flow from the PRV to the scavenging circuit increases rapidly due to the pressure in the manual breathing circuit being higher than the PEEP value and then decreases as the PRV becomes less open.

At t1, the expiration cycle ends and an inspiration cycle begins. The bag is continuously filled with fresh gas from a gas source (e.g., the gas mixer) and the bag pressure increases to the Pₘₐₓ value when the bag is compressed to push fresh gas from the bag to the patient's lungs. Furthermore, between t1 and t2, the bag pressure, and correspondingly, the P_{aw}, rise above the Pₘₐₓ value.

Initially, at t1, the PRV remains closed but the PRV diaphragm becomes displaced, e.g., opened, when the bag pressure and the P_{aw} rise above the Pₘₐₓ value and exceed the pilot pressure at the PRV. Excess pressure is vented to the scavenging circuit, as indicated by a peak in fresh gas flow from the PRV to the scavenging circuit between t1 and t2. The peak in gas flow and opening of PRV correspond to the rise in bag pressure and the P_{aw} above the Pₘₐₓ value. When the bag pressure and the P_{aw} return to the Pₘₐₓ value, the PRV diaphragm returns to a closed position and fresh gas does not flow between the PRV and the scavenging circuit.

At t2, the inspiration cycle ends and an expiration cycle begins. Each plot varies in a similar manner as during the time period between t0 and t1.

In this way, PEEP is provided to a patient during manual ventilation. By adapting a ventilation system with a manual ventilation PEEP delivery system, including an electronically-controlled TPTW valve, an electronically activated, pneumatically-controlled diaphragm valve, and a gas path for flowing gas from a bag to a scavenging circuit of the ventilation system. The manual ventilation PEEP delivery system also allows a maximum bag pressure (Pₘₐₓ) to be controlled during inspiration. Baseline pressure may be monitored and reset for each breath, thereby maintaining consistent inspiration and expiration parameters that can be readily observed by an operator and used to compare with other patient parameters. Furthermore, software algorithms for operating the manual ventilation PEEP delivery system may demand minimal modifications to already existing software algorithms for controlling mechanical ventilation at the ventilation system. The manual ventilation PEEP delivery system therefore provides a low cost, readily adaptable architecture for providing pressure control during manual ventilation.

FIGS. 6A-6C show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

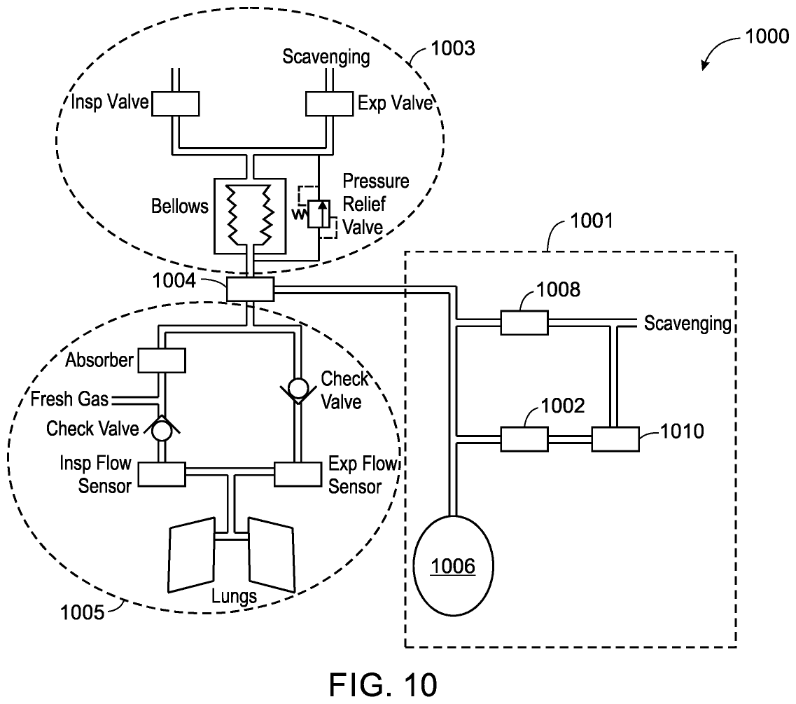

FIG. 10 shows a diagram of a ventilation system 1000 configured with a manual ventilation PEEP delivery system, according to an embodiment. In FIG. 10, some elements are numbered the same as previous figures to indicate a similar element. According to some embodiment, the ventilation system may include additional components. For example, the ventilator system 1000 may be part of an anesthesia machine.

As shown in FIG. 10, the ventilation system 1000 may include a patient circuit 1005, a mechanical ventilation circuit 1003, a manual ventilation circuit 1001, and a two-way valve 1004 connecting the circuits. The two-way valve 1004 may be switchable between a first position and a second position. The first position of the two-way valve 1004 may provide a first flow path between the manual ventilation circuit 1001 and the patient circuit 1005. The second position of the two-way valve 1004 may provide a second flow path between the mechanical ventilation circuit 1003 and the patient circuit 1005. When the two-way valve 1004 is in the first position, the second flow path may be closed by the valve and when the two-way valve is in the second position, the first flow path may be closed by the valve. The two-way valve 1004 may be similar to the BTV switch of previous embodiments. The two-way 1004 valve may be a three-port two-way valve.

The manual ventilation circuit 1001 may include a bag 1006 configured to be operated by a user providing manual ventilation to the patient. As shown in FIG. 10, a line from the bag to the two-way valve 1004 may have two branches leading to a scavenging line.

A first of the two branches may include an electrically operated cutoff valve 1002. According to an example, the cutoff valve 1002 may be actuated by a solenoid. An APL 1010 valve may be provided past the cutoff valve 1002 on the first branch. The APL valve 1010 may be a mechanical valve, such as a spring-based valve, that is operated by a user rotating a knob which increases or decreases compression of the spring. The pressure release side of the APL valve 1010 may be connected to a scavenging line.

A second of the two branches may include an electro-pneumatic APL valve 1008. According to some examples, the electro-pneumatic APL valve 1008 may be similar to PRV 534 of previous embodiment which is shown in FIGS. 6A-C. The electro-pneumatic APL valve 1008 may release pressure to the scavenging line.

Due to the mechanical properties of a mechanical APL valve 1010, such as a non-linear force profile of a spring, it may be difficult for the mechanical APL valve 1010 to provide an accurate pressure limit, especially at low pressures. An APL valve using pneumatic force, such as electro-pneumatic APL valve 1008, may be able to more accurate set a pressure limit because the pneumatic force controlling the valve can be more accurately controlled over a range of forces than mechanical components, thus more accurately controlling the pressure limit of the valve. However, drawback of the electro-pneumatically operated APL valve 1008 is that the valve cannot function when the ventilation system loses power because the pressure source and solenoid both require power, which is not an issue with the mechanically APL valve 1010.

The embodiment shown in FIG. 10 is able to utilize the positive aspects of both the mechanical APL valve 1010 and the electro-pneumatic APL valve 1008 as described below. When the ventilation system 1000 is powered on, the cutoff valve 1002 is activated, thus closing the flow path between the bag 1006 and the mechanical APL valve 1010. When the flow path to the mechanical APL valve 1010 is closed, a pressure in the line from the bag 1006 to the patient circuit 1005 is controlled by the electro-pneumatic APL valve 1008. Since the cutoff valve 1002 in only activated when the ventilation system 1000 is powered on, the electro-pneumatic APL valve 1008 will only be relied upon when the ventilation system 1000 has power and is functioning properly.

In a scenario where the ventilation system 1000 loses power, the cutoff valve 1002 will be deactivated thus opening the flow path between the bag 1006 and the mechanical APL valve 1010. In the powered off state, the electrical component (e.g. solenoid) of the electro-pneumatic APL valve 1008 will switch to a deactivated position preventing flow past the electro-pneumatic APL valve 1008 at any pressure. When the cutoff valve 1002 is open and the electro-pneumatic APL valve 1008 is closed, the pressure in the line from the bag 1006 to the patient circuit 1005 is controlled by the mechanical APL valve 1010 which is not reliant upon power from the ventilation system 1000.

According to an embodiment, the cutoff valve 1002 may be controlled by a solenoid that closes the valve when activated and the electro-pneumatic APL valve may 1008 be controlled by solenoid that closes the valve when deactivated. As such, when the ventilation system 1000 powers down, the solenoids will be deactivated do to a lack of power and will move to their default positions of opening the cutoff valve 1002 and closing the electro-pneumatic APL valve 1008 resulting in the pressure is limited by the mechanical APL 1010.

A pressure relief valve may be provided in a flow path connecting the patient side of the bellows and the expirations valve to relieve pressure on the patient side of the bellows. The pressure relief valve may be a mechanical valve, such as a spring based valve, or other known valves in the art that can be set to release pressure at a preset threshold.

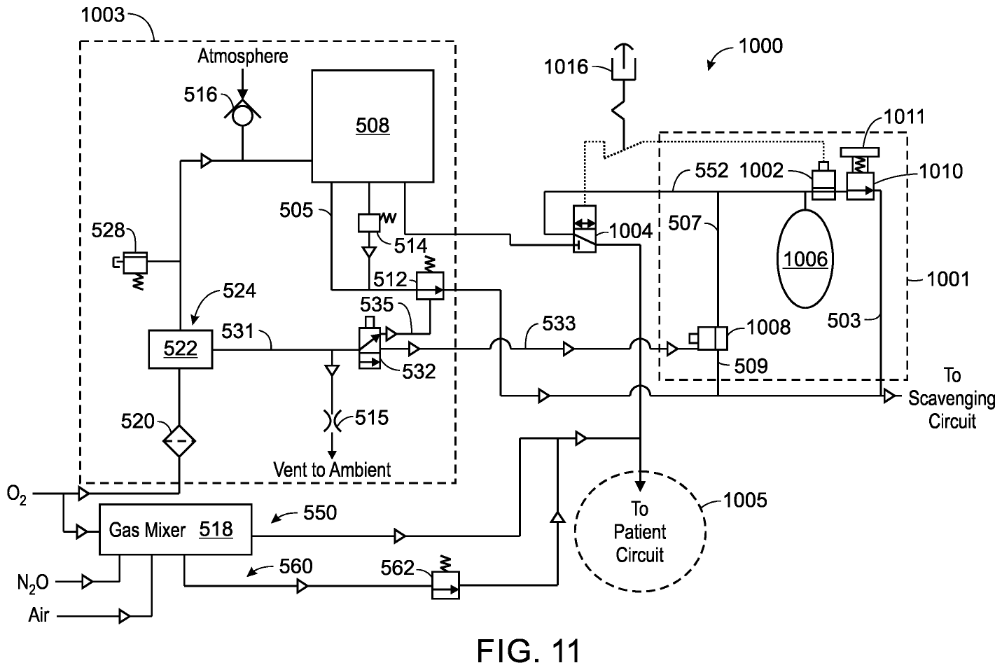

FIG. 11 shows a pneumatic diagram of a ventilation system 1000 configured with a manual ventilation PEEP delivery system, according to an embodiment. The ventilation system of FIG. 11 is similar to the system of FIG. 5 with the differences explained below.

As shown in FIG. 11, the ventilation system 1000 may include a cutoff valve 1002 between the bag 1006 and the mechanical APL valve 1010. The cutoff valve 1002 cuts off flow from the bag 1006 to the mechanical APL valve 1010 when the cutoff valve 1002 is activated. An electrically operated two-way valve 1004 connects the patient circuit to either the mechanical ventilation circuit 1003 or the manual ventilation circuit 1001, in a similar manner to the BTV valve 504 of FIG. 5. An electronic encoder 1011 may be in physical communication with the mechanical APL valve 1010 to receive and output data corresponding to the position of the mechanical APL valve 1010. For example, the electronic encoder 1011 may have a gear that meshes with gear teeth on a knob of the mechanical APL valve 1010. Due to the meshed gear teeth, when the knob of the mechanical APL vale 1010 is rotated, the electronic encoder 1011 is also rotate, thus providing data on the position of the mechanical APL valve 1010. According to an example, the electronic encoder 1011 may be an absolute optical encoder.

When the ventilation system 1000 is powered on, the cutoff valve 1002 may be activated to close the flow path between the bag 1006 and the mechanical APL valve 1010. As a result of the cutoff valve 1002 being activated, the pressure limit provided by the bag 1006 is controlled by the electro-pneumatic APL valve 1008.

When powered on, an encoder value is obtained from the electronic encoder 1011. The encoder value is provided to a controller which controls the Drive and PEEP gas control unit 522, the two-way valve 1004, the cutoff solenoid 1002, and the TPTW valve 532.

If the encoder value is above a preset value, the controller may set the two-way valve 1004 to a position in which the manual ventilation circuit 1001 is connected to the patient circuit 1005. If the encoder value is below or equal to a preset value, the controller may set the two-way valve 1004 to a position in which the mechanical ventilation circuit 1003 is connected to the patient circuit 1005. For example, the preset value may correspond to a mechanical APL valve 1010 setting of 0 cmH2O. As such, when the mechanical APL valve 1010 is set to 0 cmH2O or below, which indicates that the operator of the ventilation system 1000 intends for the patient to be ventilated by the mechanical ventilator, the controller controls the two-way valve 1004 to connect the mechanical ventilation circuit 1003 to the patient ventilation circuit 1005. According to an embodiment, the knob of the mechanical APL valve 1010 may have tactical feedback at 0 cmH2O to indicate to an operator the system is switching to mechanical ventilation and it will also have a means to secure/lock the APL knob, such as a ball plunger arrangement, to prevent accidental rotation of the APL knob.

When the encoder is below or equal to the preset value, indicating that that the operator of the ventilation system 1000 intends for the patient to be ventilated by the mechanical ventilator, the controller controls the TPTW valve 532, which may be a two-way valve, to provide pressure to the mechanical ventilation circuit 1003. When the encoder valve is above the present value, indicating that that the operator of the ventilation system 1000 intends for the patient to be ventilated by the manual ventilator, the controller controls the TPTW valve 532 to provide pressure to the electro-pneumatic APL valve to set the pressure limit of the manual ventilation circuit 1001.

The Drive and PEEP gas control unit 522 uses the encoder value to set the pressure provided to the TPTW valve 532 for controlling the electro-pneumatic APL valve 1008. That is, the pressure provided to the TPTW valve 532, which is passed to the electro-pneumatic APL valve 1008, controls the pressure limit of the manual ventilation circuit 1001.

Since the encoder 1011 tracks the position of the mechanical APL valve 1010 and the encoder value is used to set a similar pressure limit for the electro-pneumatic APL valve 1008, the pressure limit in the manual ventilation circuit 1001 will remain substantially constant when the ventilation system 1001 is powered on and off, with a possible small variation due to the different accuracies of the mechanical APL valve 1010 and electro-pneumatic APL valves 1008.

In the case of a software failure, such as a system crash, an emergency bag mode switch 1016 can be actuated to cut power to the two way valve 1004 and the cutoff valve 1002, thus switching the system to bag mode so the procedure can be continued.

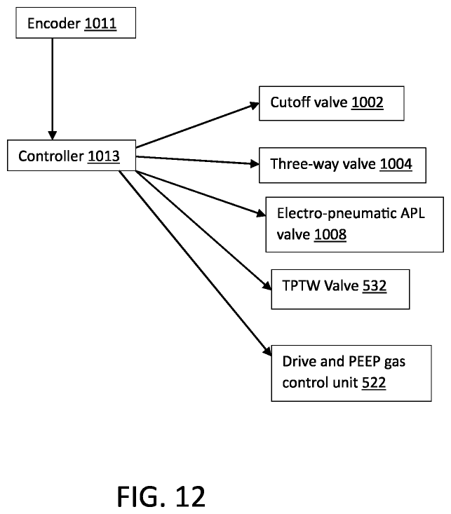

FIG. 12 shows a control diagram of a ventilation system 1000 configured with a manual ventilation PEEP delivery system, according to an embodiment. As shown in FIG. 12, the controller 1013 receives information from the encoder 1011, such as an encoder value, indicating a position of the mechanical APL valve 1010. The controller 1013 then uses the encoder value to provide control signals to the cutoff valve 1002, the two-way valve 1004, the electro-pneumatic APL valve 1008, the TPTW valve 532, and the Drive and PEEP gas control unit 522 as described above.

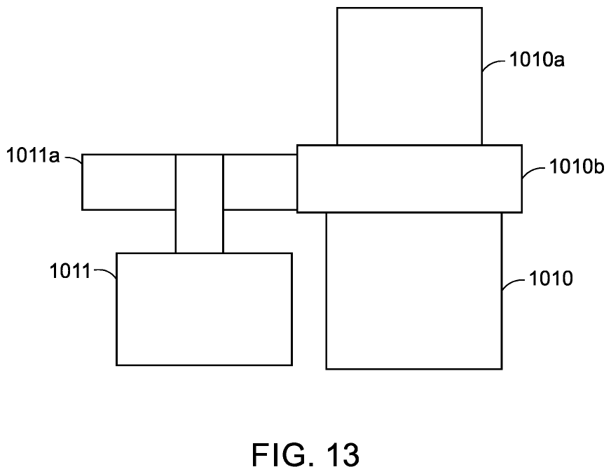

FIG. 13, shows a relationship between a mechanical APL valve 1010 and encoder 1011, according to an embodiment. As shown in FIG. 13, the mechanical APL valve 1010 may include a knob 1010a with an outwardly extending flange 1010b. An outer rim of the flange 1010b may include gear teeth. The encoder 1011 may include a gear 1011a that meshes with the gear teeth on the flange 1010b of the mechanical APL valve 1010. Due to this meshed gear relationship, when the knob 1010a of the mechanical APL 1010 is rotated, to encoder 1011 will also be rotated. The disclosure is not limited to the configuration of FIG. 13 and is intended to include other mechanism for physically transmitting rotations from the mechanical APL valve 1010 to the encoder 1011, such as a band.

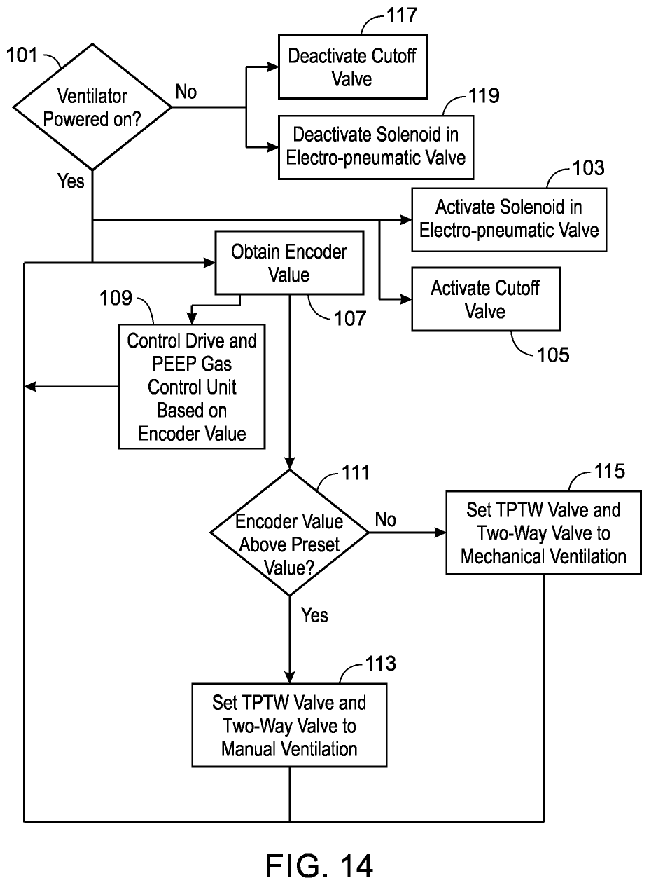

FIG. 14 is a flowchart of a process of controlling a ventilation system configured with a manual ventilation PEEP delivery system, according to an embodiment. According to an embodiment, the process may be used on the ventilation systems of FIGS. 10, 11, and 12.

At operation 101, based on whether the ventilator system is powered on, the process may progress in two directions. According to an example, an operator may power on the ventilator system to begin a procedure, such as an operation requiring anesthesia where the ventilation system is included in the anesthesia system. Powering the system on may be performed by operating a user interface such as a button, switch, or a touch sensing display. In response to the device being powered on, the process may progress to operations 104, 105, and 107.

At operation 103, the electro-pneumatic APL valve 1008 may be activated to allow the valve 1008 to control a pressure limit of the manual ventilation circuit 1001 using pneumatic pressure. According to an example, activating the electro-pneumatic APL valve 1008 may include activating a solenoid of the electro-pneumatic APL valve 1008. At operation 105, which may be performed simultaneously to operation 103, the cutoff valve 1002 may be activated to cutoff flow between the bag 1006 and the mechanical APL valve 1010. According to an example, the cutoff valve 1002 may include a solenoid that moves the cutoff valve 1002 to a cutoff position when the solenoid is activated. As such, once operations 103 and 105 have been performed, the pressure limit in the manual ventilation circuit 1003 may be controlled by the electro-pneumatic APL valve 1008.

At operation 107, an encoder valve may be obtained from the electronic encoder 1010 based on a position of the knob of the mechanical APL valve 1010.

At operation 109, the Drive and PEEP gas control unit 522 may be controlled based on the encoder value. For example, when the encoder value is above zero, the Drive and Peep gas control unit 522 may output a pressure based on the encoder value to the electro-pneumatic APL valve 1008 for setting the pressure limit of the manual ventilation circuit 1001. When the encoder valve is zero, indicating that the operator intends for the ventilation system to be mechanically controlled, the Drive and PEEP gas control unit 522 may output a drive pressure for driving the mechanical ventilation circuit 1003.

At operation 111, the process may determine whether the encoder value is above a preset value. According to an example, the preset value may be associated with the knob of the mechanical APL valve 1010 being set to 0.0 mmH2O. Based on the encoder value being above the preset value, the process may progress to operation 113.

At operation 113, the TPTW valve 532 may be set to manual ventilation and the two-way valve 1004 may be set to manual ventilation. In manual ventilation, the three way-valve 1004 may connect the manual ventilation circuit 1001 to the patient circuit 1005 and the TPTW valve 532 may direct pressure provided by the Drive and Peep gas control unit 522 to the electro-pneumatic APL valve 1008.

Based on the encoder value being at or below the preset value, the process may progress to operation 115. At operation 115, the TPTW valve 532 may be set to mechanical ventilation and the two-way valve 1004 may be set to mechanical ventilation. In mechanical ventilation, the three way-valve 1004 may connect the mechanical ventilation circuit 1003 to the patient circuit 1005 and the TPTW valve 532 may direct pressure provided by the Drive and Peep gas control unit 522 to the mechanical ventilation circuit 1003.

Based on the ventilator not being powered on at operation 101, the process may progress to operations 117 and 119. At operation 117, the electro-pneumatic APL valve 1008 may be deactivated to close the valve 1008 to scavenging. According to an example, deactivating the electro-pneumatic APL valve 1008 may include deactivating the solenoid of the electro-pneumatic valve 1008 which forces closed a flow path to scavenging. At operation 119, which may be performed simultaneously to operation 117, cutoff valve 1008 may be deactivated to open flow between the bag 1006 and the mechanical APL valve 1010. According to an example, the solenoid of the cutoff valve 1002 may allow flow through the cutoff valve 1002 when the solenoid is deactivated. As such, once operations 117 and 119 have been performed, the pressure limit in the manual ventilation circuit 1001 may be controlled by the mechanical APL. According to an example, due to the physical characteristics of solenoids, operations 117 and 119 may be performed by merely cutting power to the solenoids of the electro-pneumatic APL valve 1008 and the cutoff valve 1002.

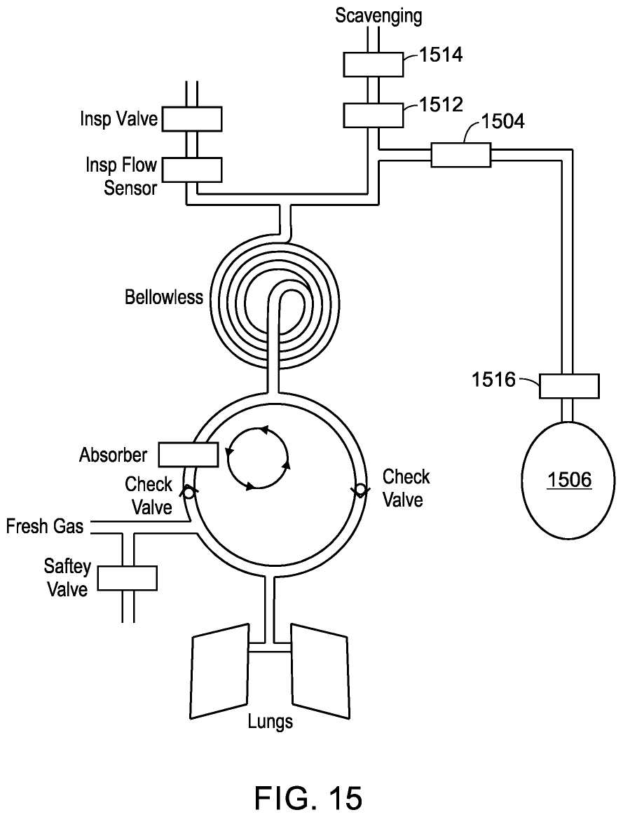

FIG. 15 shows a diagram of a bellowless ventilation system configured with a manual ventilation PEEP delivery system, according to an embodiment. The bellowless ventilation system may function the same as ventilation system 1000 with the expiration valve 1512 of the bellowless system acting as the PEEP valve during expiration and the APL valve during inspiration.

As shown in FIG. 15, the ventilation circuit includes a bellowless design. In a bag mode, the patient may be ventilated by member of the care team operating bag 1506. A bag flow sensor 1516 may detect flow provided by the bag 1506. A bag to vent cutoff valve 1504 may cutoff flow between the bag and the patient. The cutoff valve 1504 may be set to a closed position for mechanical ventilation and an open position for bag ventilation.

Expiration valve 1512 may be similar to electro-pneumatic APL valve 1008. When bag ventilation is being performed, the expiration valve 1512 may maintain PEEP during expiration in a similar manner as discussed above, and the expiration valve 1512 may act as an APL valve during inspiration in a similar manner as discussed above. An expiration flow sensor 1514 may be provided between the expiration valve 1512 and scavenging to detect expiration flow to scavenging.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

The disclosure also provides support for a method for operating a ventilation system in a manual ventilation mode, comprising: activating an electronically-controlled valve and a pneumatically-controlled valve to provide a pilot pressure to the pneumatically-controlled valve to deliver a target positive end expiratory pressure (PEEP) from a gas control unit during expiration, adjusting the pilot pressure based on a comparison of a patient airways pressure to the target PEEP. In a first example of the method, activating the electronically-controlled valve includes energizing an electromagnet to control a position of a solenoid of the electronically-controlled valve. In a second example of the method, optionally including the first example, activating the pneumatically-controlled valve includes retracting a solenoid away from a diaphragm of the pneumatically-controlled valve, and wherein the pilot pressure is communicated to a first face of the diaphragm and a pressure from a bag and/or breathing system of the ventilation system is communicated to a second face of the diaphragm, the second face opposite of the first face. In a third example of the method, optionally including one or both of the first and second examples, when the pressure from the bag and/or breathing system is less than or equal to the pilot pressure, a gas flow from the bag to a scavenging circuit of the ventilation system is blocked, and wherein when the pressure from the bag and/or breathing system is greater than the pilot pressure, the diaphragm is displaced and the gas flow from the bag to the scavenging circuit is enabled. In a fourth example of the method, optionally including one or more or each of the first through third examples, the pilot pressure is set to a target maximum pressure of a bag of the ventilation system during an inspiration cycle of the ventilation system and set to the target PEEP during an expiration cycle of the ventilation system. In a fifth example of the method, optionally including one or more or each of the first through fourth examples, the pilot pressure is controlled by adjusting a pilot gas flow from the gas control unit. In a sixth example of the method, optionally including one or more or each of the first through fifth examples, adjusting the pilot pressure includes adjusting a position of a valve of the gas control unit at a beginning of each expiration cycle to increase the pilot pressure when the patient airways pressure is less than the target PEEP or to decrease the pilot pressure when the patient airways pressure is greater than the target PEEP, and wherein the pilot pressure is adjusted for each breath of a patient.

The disclosure also provides support for a ventilation system, comprising: a pneumatically-controlled valve arranged in a path of pilot gas flow between a bag and a scavenging circuit of the ventilation system, the pneumatically-controlled valve adjusted based on a pilot pressure directed thereto by an electronically-controlled valve to provide a positive end expiratory pressure (PEEP) to a patient during manual ventilation of the patient. In a first example of the system, the pneumatically-controlled valve includes a diaphragm and solenoid, and wherein the solenoid blocks displacement of the diaphragm when the pneumatically-controlled valve is deactivated, and wherein the solenoid is retracted away from the diaphragm when the pneumatically-controlled valve is activated. In a second example of the system, optionally including the first example, when the diaphragm is not displaced, the pneumatically-controlled valve closes the path of pilot gas flow between the bag and the scavenging circuit and when the diaphragm displaced, the pneumatically-controlled valve is open to the path of pilot gas flow. In a third example of the system, optionally including one or both of the first and second examples, the electronically-controlled valve is a three-port, two-way solenoid valve, and wherein the electronically-controlled valve is positioned between a Drive and PEEP gas control unit and the pneumatically-controlled valve, the Drive and PEEP gas control unit configured to regulate a pilot gas delivered to the pneumatically-controlled valve from a gas source. In a fourth example of the system, optionally including one or more or each of the first through third examples, the electronically-controlled valve includes a first port for receiving the pilot gas from the Drive and PEEP gas control unit, a second port for directing the pilot gas to an exhalation valve, and a third port for directing the pilot gas to the pneumatically-controlled valve. In a fifth example of the system, optionally including one or more or each of the first through fourth examples, the exhalation valve is included in a mechanical ventilation circuit of the ventilation system. In a sixth example of the system, optionally including one or more or each of the first through fifth examples, a position of a solenoid of the electronically-controlled valve is adjusted between directing a flow of the pilot gas out of the second port during mechanical ventilation and directing a flow of the pilot gas out of the third port during manual ventilation. In a seventh example of the system, optionally including one or more or each of the first through sixth examples, the pilot pressure is moderated to provide the PEEP during expiration cycles and to provide a maximum bag pressure during inspiration cycles.

The disclosure also provides support for a method for a ventilation system, comprising: responsive to adjustment of the ventilation system to operation in a manual ventilation mode, flowing a pilot gas from an electronically-controlled three-port, two-way (TPTW) valve to a pneumatically-controlled pressure relief valve (PRV) during an expiration cycle to deliver a positive end expiratory pressure (PEEP) to a patient, and responsive to adjustment of the ventilation system to operation in a mechanical ventilation mode, blocking a flow of the pilot gas to the pneumatically-controlled pressure relief valve and maintaining the PEEP based on a PEEP setting of the manual ventilation mode. In a first example of the method, the method further comprises: activating the TPTW valve and the PRV upon adjustment of the ventilation system to operation in the manual ventilation mode by energizing respective electro-magnets of the TPTW valve and the PRV in response to adjusting a bag-to-ventilation switch. In a second example of the method, optionally including the first example, the PRV is de-energized and a solenoid of the PRV blocks gas flow between a bag and a scavenging circuit of the ventilation system when power to the ventilation system is lost. In a third example of the method, optionally including one or both of the first and second examples, the adjustment of the ventilation system to operation in the manual ventilation mode includes receiving one or more of the PEEP setting and a maximum bag pressure setting from an operator at a user interface of the ventilation system. In a fourth example of the method, optionally including one or more or each of the first through third examples, the method further comprises: in response to detection of a lack of inspiration or expiration of the patient, adjusting or maintaining a pressure of a manual circuit of the ventilation system at the PEEP.

The disclosure also provides support for a method for controlling a medical ventilator, the medical ventilator comprising a manual ventilation circuit, a mechanical ventilation circuit, and a patient circuit, the method includes: obtaining an encoder value based on a pressure limit of a mechanical adjustable pressure limit valve (APLV) in the manual ventilation circuit; controlling a pressure limit of an electro-pneumatic APLV in the manual ventilation circuit based on the encoder value, the electro-pneumatic APLV being pneumatically connected in parallel to the mechanical APLV.

The method may further include based on the medical ventilator being powered on: controlling a shutoff valve to shut off flow between a bag of the manual ventilation circuit and the mechanical APLV; and controlling a pressure limit of the electro-pneumatic APLV based on a pneumatic pressure.

The method may further include, based on the medical ventilator being powered off: controlling the shutoff valve to allow flow between the bag of the manual ventilation circuit and the mechanical APLV; and controlling the electro-pneumatic APLV to be held in shutoff state preventing pressure release.

Controlling the shutoff valve to shutoff flow may include activating a solenoid of the cutoff valve, and controlling the electro-pneumatic APLV to control a pressure based on a pneumatic pressure may include activating a solenoid of the electro-pneumatic APLV.

The method may further include, based on the encoder value being above a preset valve, controlling the medical ventilator to pneumatically connect the manual ventilation circuit and the patient circuit for providing manual ventilation to the patient.

The method may further include, based on the encoder value being above a preset value, controlling a two-way valve to provide pressure to the electro-pneumatic APLV.

A pressure level of the provided pressure may be based on the encoder value.

The method may further include, based on the encoder valve being below or equal to the preset valve, controlling the medical ventilator to pneumatically connect the mechanical ventilation circuit and the patient circuit for providing mechanical ventilation to the patient.

The method may further include, based on the encoder value being below or equal to a preset value, controlling the two-way valve to provide pressure to the mechanical ventilation circuit.

The disclosure also provides support for a medical ventilator which may include: a manual ventilation circuit configured to provide manual ventilation to a patient, the manual ventilation circuit including: a bag configured to be operated by a user; a mechanical adjustable pressure limit valve (APLV); a electro-pneumatic APLV pneumatically connected in parallel with the mechanical APLV; an encoder configured to output an encoder value based on a pressure limit of the mechanical APLV; and a shutoff valve between the bag and the mechanical APLV; a mechanical ventilation circuit configured to provide mechanical ventilation to a patient through bellows; a patient circuit configured to deliver gas to and remove gas from a patient's lungs; a memory storing instructions; and at least one processor configured to execute the instructions to: control a pressure limit of the electro-pneumatic APLV based on the encoder value.

The at least one processor may be further configured to execute the instructions to, based on the medical ventilator being powered on: control the shutoff valve to shut off flow between the bag and the mechanical APLV; and apply a pressure to the electro-pneumatic APLV to pneumatically control a pressure limit of the electro-pneumatic APLV.

The at least one processor may be further configured to execute the instructions to, based on the medical ventilator being powered off: control the shutoff valve to allow flow between the bag and the mechanical APLV; and control the electro-pneumatic APLV to be held in shutoff state preventing pressure release.

Controlling the shutoff valve to shutoff flow may include activating a solenoid of the cutoff valve, and controlling the electro-pneumatic APLV to control a pressure based on a pneumatic pressure may include activating a solenoid of the electro-pneumatic APLV.

The at least one processor may be further configured to execute the instructions to, based on the encoder value being above a preset valve, control the medical ventilator to pneumatically connect the manual ventilation circuit and the patient circuit for providing manual ventilation to the patient.

The at least one processor may be further configured to execute the instructions to, based on the encoder value being above a preset value, control a two-way valve to provide pressure to the electro-pneumatic APLV.

A pressure level of the provided pressure may be based on the encoder value.

The at least one processor may be further configured to execute the instructions to, based on the encoder valve being below or equal to the preset valve, control the medical ventilator to pneumatically connect the mechanical ventilation circuit and the patient circuit for providing mechanical ventilation to the patient.

The at least one processor may be further configured to execute the instructions to, based on the encoder value being below or equal to a preset value, control the two-way valve to provide pressure to the mechanical ventilation circuit.

The disclosure also provides support for a medical ventilator system which may include: a manual ventilation circuit including: a bag configured to be operated by a user; and a user adjustable encoder configured to output an encoder value based on a pressure limit of a first adjustable pressure limit valve (APLV) in the manual ventilation circuit; a mechanical ventilation circuit comprising an electronically controlled mechanical ventilator; a patient circuit configured to deliver gas to and remove gas from a patient's lungs; a first flow path provided between the manual ventilation circuit and the patient circuit; a second flow path provided between the mechanical ventilation circuit and the patient circuit; a first electronically controlled two-way valve having a first position providing the first flow path and a second position providing the second flow path; a memory storing instructions; and at least one processor configured to execute the instruction to: control a maximum pressure limit in the first flow path based on the encoder value; control the first two-way valve based on the encoder value.

The medical ventilator may further include a second electronically controlled two-way valve having a first position providing a third flow path between a controlled pressure source and the mechanical ventilation circuit and a second position providing a fourth flow path between the controlled pressure source and the first APLV.

The pressure limit of the first APLV may be controlled by pressure in the fourth flow path.

The medical ventilator may further include a second APLV provided in a fifth flow path between the bag and a scavenging line, the second APLV being configured to control a pressure limit in the fifth flow path. The encoder value may be based on a position of the second APLV.

Rotating a knob of the second APLV may cause the encoder value to change.

The medical ventilator may further include an electronically controlled cutoff valve provided in a sixth flow path between the bag and the second APLV, the cutoff valve being configured to cutoff flow in the sixth flow path when activated.

The processor may be further configured to execute the instructions to activate the cutoff valve in response to the medical ventilator powering on.

The processor may be further configured to execute the instructions to deactivate the cutoff valve in response to the medical ventilator powering off.

The processor may be further configured to execute the instructions to control the first two-way valve to be in the first position based on the encoder value being equal to or below a preset value and control the first two-way valve to be in the second position based on the encoder value being greater than the preset value.

The processor may be further configured to execute the instructions to control the second two-way valve to be in the first position based on the encoder value being equal to or below a preset value and control the second two-way valve to be in the second position based on the encoder value being greater than the preset value.

A pressure limit of the first APLV may be controlled pneumatically and a pressure limit of the second APLV is controlled mechanically.

The first APLV may be an electro-pneumatic valve.

The processor may be further configured to execute the instructions to control a solenoid of the first APLV to deactivate in response to the medical ventilator powering off, wherein deactivate the solenoid locks the first APLV is a closed position that does not regulate pressure.

The processor may be further configured to execute the instructions to control the solenoid of the first APLV to activate in response to the medical ventilator powering on, wherein activating the first solenoid unlocks the first APLV for pressure regulation.

The manual ventilation circuit may further include a second APLV. The second APLV may be mechanically controlled.

The first APLV and the second APLV may be pneumatically connected in parallel.

A pressure limit of the first APLV may be based on a pressure limit of the second APLV.

The medical ventilator may further include an encoder configured to output an encoder value corresponding to a pressure limit of the second APLV, wherein the processor is further configured to execute the instruction to control a pressure provided to the first APLV based on the encoder value.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method for controlling a medical ventilator, the medical ventilator comprising a manual ventilation circuit, a mechanical ventilation circuit, and a patient circuit, the method comprising:
obtaining an encoder value based on a pressure limit of a mechanical adjustable pressure limit valve (APLV) in the manual ventilation circuit;
controlling a pressure limit of an electro-pneumatic APLV in the manual ventilation circuit based on the encoder value, the electro-pneumatic APLV being pneumatically connected in parallel to the mechanical APLV.

2. The method of claim 1, further comprising, based on the medical ventilator being powered on:
controlling a shutoff valve to shut off flow between a bag of the manual ventilation circuit and the mechanical APLV; and
controlling a pressure limit of the electro-pneumatic APLV based on a pneumatic pressure.

3. The method of claim 2, further comprising, based on the medical ventilator being powered off:
controlling the shutoff valve to allow flow between the bag of the manual ventilation circuit and the mechanical APLV; and
controlling the electro-pneumatic APLV to be held in shutoff state preventing pressure release.

4. The method of claim 2, wherein controlling the shutoff valve to shutoff flow comprises activating a solenoid of the cutoff valve, and
wherein controlling the electro-pneumatic APLV to control a pressure based on a pneumatic pressure comprises activating a solenoid of the electro-pneumatic APLV.

5. The method of claim 1, further comprising, based on the encoder value being above a preset valve, controlling the medical ventilator to pneumatically connect the manual ventilation circuit and the patient circuit for providing manual ventilation to the patient.

6. The method of claim 5, further comprising, based on the encoder value being above a preset value, controlling a two-way valve to provide pressure to the electro-pneumatic APLV.

7. The method of claim 6, wherein a pressure level of the provided pressure is based on the encoder value.

8. The method of claim 5, further comprising, based on the encoder valve being below or equal to the preset valve, controlling the medical ventilator to pneumatically connect the mechanical ventilation circuit and the patient circuit for providing mechanical ventilation to the patient.

9. The method of claim 8, further comprising, based on the encoder value being below or equal to a preset value, controlling the two-way valve to provide pressure to the mechanical ventilation circuit.

10. A medical ventilator comprising:
a manual ventilation circuit configured to provide manual ventilation to a patient, the manual ventilation circuit comprising:
a bag configured to be operated by a user;
a mechanical adjustable pressure limit valve (APLV);
a electro-pneumatic APLV pneumatically connected in parallel with the mechanical APLV;
an encoder configured to output an encoder value based on a pressure limit of the mechanical APLV; and
a shutoff valve between the bag and the mechanical APLV;
a mechanical ventilation circuit configured to provide mechanical ventilation to a patient through bellows;
a patient circuit configured to deliver gas to and remove gas from a patient's lungs;
a memory storing instructions; and
at least one processor configured to execute the instructions to:
control a pressure limit of the electro-pneumatic APLV based on the encoder value.

11. The medical ventilator of claim 10, wherein the at least one processor is further configured to execute the instructions to, based on the medical ventilator being powered on:
control the shutoff valve to shut off flow between the bag and the mechanical APLV; and
apply a pressure to the electro-pneumatic APLV to pneumatically control a pressure limit of the electro-pneumatic APLV.

12. The medical ventilator of claim 11, wherein the at least one processor is further configured to execute the instructions to, based on the medical ventilator being powered off:
control the shutoff valve to allow flow between the bag and the mechanical APLV; and
control the electro-pneumatic APLV to be held in shutoff state preventing pressure release.

13. The medical ventilator of claim 11, wherein controlling the shutoff valve to shutoff flow comprises activating a solenoid of the cutoff valve, and
wherein controlling the electro-pneumatic APLV to control a pressure based on a pneumatic pressure comprises activating a solenoid of the electro-pneumatic APLV.

14. The medical ventilator of claim 10, wherein the at least one processor is further configured to execute the instructions to, based on the encoder value being above a preset valve, control the medical ventilator to pneumatically connect the manual ventilation circuit and the patient circuit for providing manual ventilation to the patient.

15. The medical ventilator of claim 14, wherein the at least one processor is further configured to execute the instructions to, based on the encoder value being above a preset value, control a two-way valve to provide pressure to the electro-pneumatic APLV.

16. The medical ventilator of claim 15, wherein a pressure level of the provided pressure is based on the encoder value.

17. The medical ventilator of claim 14, wherein the at least one processor is further configured to execute the instructions to, based on the encoder valve being below or equal to the preset valve, control the medical ventilator to pneumatically connect the mechanical ventilation circuit and the patient circuit for providing mechanical ventilation to the patient.

18. The medical ventilator of claim 17, wherein the at least one processor is further configured to execute the instructions to, based on the encoder value being below or equal to a preset value, control the two-way valve to provide pressure to the mechanical ventilation circuit.

19. A medical ventilator system comprising:
a manual ventilation circuit comprising:
a bag configured to be operated by a user; and
a user adjustable encoder configured to output an encoder value based on a pressure limit of a first adjustable pressure limit valve (APLV) in the manual ventilation circuit;
a mechanical ventilation circuit comprising an electronically controlled mechanical ventilator;
a patient circuit configured to deliver gas to and remove gas from a patient's lungs;
a first flow path provided between the manual ventilation circuit and the patient circuit;
a second flow path provided between the mechanical ventilation circuit and the patient circuit;
a first electronically controlled two-way valve having a first position providing the first flow path and a second position providing the second flow path;
a memory storing instructions; and
a processor configured to execute the instruction to:
control a maximum pressure limit in the first flow path based on the encoder value;
control the first two-way valve based on the encoder value.

20. The medical ventilator of claim 19, further comprising a second electronically controlled two-way valve having a first position providing a third flow path between a controlled pressure source and the mechanical ventilation circuit and a second position providing a fourth flow path between the controlled pressure source and the first APLV, wherein the pressure limit of the first APLV is controlled by pressure in the fourth flow path.

21. The medical ventilator of claim 19, further comprising a second APLV provided in a fifth flow path between the bag and a scavenging line, the second APLV being configured to control a pressure limit in the fifth flow path, wherein the encoder value is based on a position of the second APLV.

22. The medical ventilator of claim 21, further comprising an electronically controlled cutoff valve provided in a sixth flow path between the bag and the second APLV, the cutoff valve being configured to cutoff flow in the sixth flow path when activated, wherein the processor is further configured to execute the instructions to activate the cutoff valve in response to the medical ventilator powering on.

23. The medical ventilator of claim 21, wherein the processor is further configured to execute the instructions to control the first two-way valve to be in the first position based on the encoder value being equal to or below a preset value and control the first two-way valve to be in the second position based on the encoder value being greater than the preset value.

24. The medical ventilator of claim 21, wherein the processor is further configured to execute the instructions to control the second two-way valve to be in the first position based on the encoder value being equal to or below a preset value and control the second two-way valve to be in the second position based on the encoder value being greater than the preset value.

25. The medical ventilator of claim 21, wherein a pressure limit of the first APLV is controlled pneumatically and a pressure limit of the second APLV is controlled mechanically.
